(19) United States
(12) Patent Application Publication
(10) Pub. No.: US 2011/0158979 A1
(43) Pub. Date: Jun. 30, 2011

(54) GENETIC COMPONENT OF COMPLICATIONS IN TYPE 2 DIABETES

(75) Inventors: Pavel Hamet, Ville Mont-royal (CA); Johanne Tremblay, Ville Mont-royal (CA); Ondrej Seda, Boucherville (CA); Stephen Macmahon, New South Wales (AU); John Chalmers, New South Wales (AU)

(73) Assignee: PROGNOMIX, INC., Montreal (CA)

(21) Appl. No.: 12/997,621

(22) PCT Filed: Jun. 15, 2009
(86) PCT No.: PCT/IB2009/006638

§ 371 Date: Mar. 14, 2011

Related U.S. Application Data
(60) Provisional application No. 61/061,389, filed on Jun. 13, 2008.

(51) Int. Cl. A61K 39/395; C12Q 1/68; C40B 30/00; C40B 40/06; A61K 31/7105; A61P 3/10

(52) U.S. Cl. 424/130.1; 435/6.12; 506/7; 506/16; 435/6.11; 435/6.1; 514/44 A

(57) ABSTRACT

The invention provides with means to predict, in subjects affected by type 2 diabetes (T2D), the probability of developing complications related to the disease. The invention involves 1) use of genetic features (SNPs, STRs, or other genomic markers) together with other chromosomal features and phenotypic information to establish a patient profile specifically developed for prediction of complications of T2D 2) use of a set of SNPs allowing to discriminate between individuals according to their descent. The invention also provides with methods for characterizing and selecting, within a population of subjects with type-2 diabetes, subjects that are suited for clinical trials based on the identification of one or more genetic features. Also described are combinations and kits for carrying out the above-described methods.

This application claims the benefit of U.S. provisional Application No. 61/061,389, filed on Jun. 13, 2008, the disclosure of which is incorporated herein by reference in its entirety.

The invention provides means and methods to predict, in subjects affected by type II diabetes (T2D), the probability of developing complications related to the disease.

The invention further provides with methods for characterizing and selecting, within a population of subjects with type II diabetes (T2D), subjects that are suited for clinical trials based on the identification of one or more genetic features, which are single nucleotide polymorphisms (SNPs), short tandem repeats (STRs) and/or other genomic markers in addition to the currently used biological markers of high cardiovascular risk. The invention further involves characterizing these subjects based on the probability of developing complications related to T2D, such as, myocardial infarction, stroke albuminuria and/or declining glomerular filtration. Also described are combinations and kits for carrying out the above-described methods.

Diabetes mellitus is a heterogeneous group of metabolic diseases which is characterized by elevated blood glucose levels and increased morbidity. The endocrine cells of the pancreas which synthesize insulin and other hormones are involved in the pathogenesis of diabetes. Both genetic and environmental factors contribute to its development. The most common form is T2D, which is characterized by defects in both insulin secretion and insulin action. In contrast, type I diabetes results from autoimmune destruction of the insulin-producing beta cells of the pancreas. Monogenic forms of diabetes account for less than 5% of the cases and are usually caused by mutations in genes associated with maturity-onset diabetes of the young (MODY), insulin gene and insulin receptor gene.

T2D is a heterogeneous disease resulting from the interaction of environmental factors such as obesity or sedentary lifestyle, with variety of diabetogenic genes. Abnormal glucose homeostasis occurs when either insulin sensitivity or insulin secretion or both are altered. An early finding in this development is insulin resistance, defined as impaired insulin-mediated glucose clearance in insulin-sensitive tissues (skeletal muscle, liver and adipose tissue). Elevation of glucose levels triggers beta-cells to produce and secrete more insulin, which compensates for the disturbance in glucose homeostasis. The duration of hyperglycemia-hyperinsulinemia state depends on insulin secretory capacity, mass and apoptosis rate of beta-cells. Furthermore, beta-cells can loose their insulin secretion capacity because of glucose toxicity or other reasons. When cells fail to compensate for insulin resistance blood glucose concentration increases. Thus, over time subclinical hyperglycemia tends to progress to impaired glucose tolerance and further to T2D.

The causes of T2D are multi-factorial and include both genetic and environmental elements that affect beta cell function and insulin sensitivity of peripheral tissues (muscle, liver, adipose tissue, pancreas). Although there is considerable debate as to the relative contributions of beta-cell dysfunction and reduced insulin sensitivity to the pathogenesis of diabetes, it is generally agreed that both of these factors play important roles. Both impaired insulin secretion and insulin action cause the development of T2D. Insulin resistance is an early feature in the pathophysiology of T2D.

No major single gene explaining the development of T2D has been identified. There were several studies attempting to predict T2D based on limited number of SNPs (up to 18) [Lyssenko, V, P Almgren, D Anevski et al.: Genetic prediction of future type 2 diabetes. PLoS Med 2, e345 (2005); Meigs, J B, P Shrader, L M Sullivan et al.: Genotype score in addition to common risk factors for prediction of type 2 diabetes. N Engl J Med 359, 2208-19 (2008); Cauchi, S, D Meyre, E Durand et al.: Post genome-wide association studies of novel genes associated with type 2 diabetes show gene-gene interaction and high predictive value. PLoS ONE 3, e2031 (2008). Miyake, K, W Yang, K Hara et al.: Construction of a prediction model for type 2 diabetes mellitus in the Japanese population based on 11 genes with strong evidence of the association. J Hum Genet 54, 236-41 (2009); Lin, X, K Song, N Lim et al.: Risk prediction of prevalent diabetes in a Swiss population using a weighted genetic score—the CoLaus Study. Diabetologia 52, 600-8 (2009)] but the reached predictive power was generally limited. Indeed, the mathematical models reflecting the presumable allele frequencies and risk effects estimate that for successful prediction, hundreds of SNPs will be necessary [(Kraft, P, D J Hunter: Genetic Risk Prediction—Are We There Yet? N Engl J Med 360, 1701-1703 (2009)].

Complications Associated with Diabetes

Over 90% of people diagnosed with diabetes have T2D, which carries a number of potential complications. These complications currently add very significantly to the cost of treating diabetes, because there is no reliable way to determine which patients are likely to develop such difficulties. Half of the people affected by T2D die from complications resulting from the disease.

Such complications include, but are not limited to:

Cardiovascular Disease

Cardiovascular disease is the overwhelming cause of diabetes-related deaths. With the risk for stroke or myocardial infarction elevated by 2 to 4 times in persons with diabetes, a 65% majority of deaths among people with diabetes occurs from heart disease or stroke, considered as major macrovascular complications.

Diabetic Nephropathy

End-stage renal disease (ESRD) occurs when the kidneys cease to function, which ultimately leads to the need for a transplant or regular dialysis, both extremely costly procedures. Diabetes is responsible for 43% of the cases of ESRD as a consequence of microvascular damage of the kidney.

Diabetic Retinopathy

Diabetes is also the leading cause of blindness in people aged 20-74. Diabetic retinopathy is considered as one type of microvascular complication and is responsible for over 24,000 cases of blindness in the United States.

Diabetic Neuropathy

It is estimated that over 70% of people with diabetes may also suffer from nervous system damage, causing impaired sensation or pain in the feet or hands, slowed digestion of food in the stomach, carpal tunnel syndrome, and other nerve problems. In the severe cases of diabetic neuropathy, usually combined with peripheral vessel macro and microvascular disease, patients may have to undergo lower-extremity amputations.

Dental disease, complications of pregnancy, coma, and acute susceptibility to opportunistic infectious diseases are also costly diabetes-related diseases.

Drugs designed to prevent or stabilize complications are extremely costly. Because of the debilitating effects of diabetes-related complications, healthcare professionals are forced to prescribe costly medications to diabetes patients to protect them against developing these complications without having any efficient and reliable mean to predict those patients who will develop these complications and the efficiency of these treatments.

There is a need for assays capable of identifying among T2D patients those who are at risk of developing complications and those who would benefit from the various treatments available. The present invention provides with means to develop such assays and to utilize them in a clinical and medical environment.

Polymorphisms

DNA polymorphisms provide an efficient way to study the association of genes and diseases by analysis of linkage and linkage disequilibrium. With the sequencing of the human genome a myriad of hitherto unknown genetic polymorphisms among people have been detected. Most common among these are the single nucleotide polymorphisms, also called SNPs, of which there are known several millions. Other examples are short tandem repeat polymorphisms (STR), variable number of tandem repeat polymorphisms (VNTR), insertions, deletions and block modifications. Tandem repeats (STR or VNTR) often have multiple different alleles (variants) in population, whereas the other groups of polymorphisms usually have just two alleles. Some of these genetic polymorphisms play a direct role in the biology of the individuals, including their risk of developing disease, but the virtue of the majority is that they can serve as markers for the surrounding DNA. The relationship of an allele of one sequence polymorphism with particular alleles of other sequence polymorphisms in the surrounding is due to phenomenon called genetic linkage. Linkage arises because large parts of chromosomes are passed unchanged from parents to offspring, so that minor regions of a chromosome tend to flow unchanged from one generation to the next and also to be similar in different branches of the same family. Linkage is gradually eroded by recombination occurring in the germline cells, but typically operates over multiple generations and distances of a number of million bases in the DNA.

Linkage disequilibrium deals with whole populations and has its origin in the (distant) forefather in whose DNA a new sequence polymorphism arose. The immediate surroundings in the DNA of the forefather will tend to stay with the new allele and propagate together to the offspring for many generations. Recombination and changes in the composition of the population will again erode the association, but the new allele and the alleles of any other polymorphism nearby will often be partly associated among unrelated humans even today. A crude estimate suggests that alleles of sequence polymorphisms with distances less than 10000 bases in the DNA will have tended to stay together since modern man arose. Linkage disequilibrium in limited populations, for instance Europeans, often extends over longer distances, e.g. over more than 1,000,000 bases. This can be the result of newer mutations, but can also be a consequence of one or more “bottlenecks” with small effective population sizes and considerable inbreeding in the history of the current population. Two obvious possibilities for “bottlenecks” in Europeans are the exodus from Africa and the repopulation of Europe after the last ice age. A number of polymorphisms have been associated with induction of exocrine pancreatic dysfunction and/or diabetes. Some of the identified polymorphisms have been suggested in patent literature as useful in diagnosis of diabetes (see for example WO9321343 related to polymorphisms in GCK gene, and WO0023591 related to polymorphism in ZSIG49 gene).

Up to date, several genomic regions or individual genetic variants have been found to be linked or associated to the phenotypes closely related to diabetic complications.

Linkage Studies

In a recent study (Diabetes. 2008 January; 57(1):235-43), a genome-wide scan for estimated glomerular filtration rate was performed in multi-ethnic diabetic populations (the Family Investigation of Nephropathy and Diabetes (FIND)) using 404 STR markers. For all ethnicities combined, strong evidence for linkage was observed on chromosomes 1q43, 7q36.1, 8q13.3 and 18q23.3. Mexican-American families, who comprised the major ethnic subpopulation in FIND, contributed to linkage on chromosomes 1q43, 2p13.3, 7q36.1, 8q13.3, and 18q23.3, whereas African-American and American-Indian families displayed linkage peaks on chromosomes 11p15.1 and 15q22.3, respectively. Also in FIND study (Diabetes. 2007 June; 56(6):1577-85), the strongest evidence of linkage to the diabetic nephropathy trait was on chromosomes 7q21.3, 10p15.3, 14q23.1, and 18q22.3. In ACR (883 diabetic sibling pairs), the strongest linkage signals were on chromosomes 2q14.1, 7q21.1, and 15q26.3. These results confirm regions of linkage to diabetic nephropathy on chromosomes 7q, 10p, and 18q from prior reports. In Mexican Americans, Puppala al. (Diabetes. 2007 November; 56 (11):2818-28) found a linkage of glomerular filtration rate to a region on chromosome 2q near marker D2S427 (corrected LOD score 3.3), influenced by genotype by diabetes interaction.

Association Studies

A number of genes and genetic polymorphisms were tested for their association to diabetic nephropathy, either because of their reported relevance in metabolic and signaling pathways connected to pathophysiology of diabetic complications (functional candidates) or combination of the former with their genomic position under peak of ascertained linkage (positional candidates), or as a result of genome-wide association studies. Genes for which an association was found with diabetic nephropathy include 5,10-methylenetetrahydrofolate reductase (MTHFR), natriuretic peptide precursor A (NPPA), solute carrier family 2 member 1 (facilitated glucose transporter SLC2A1), lamin A/C (LMNA), retinoid X receptor gamma (RXRG), interleukin 1 receptor antagonist (IL1RN), ghrelin/obestatin preprohormone (GHRL), peroxisome proliferator-activated receptor gamma (PPARG), chemokine receptor 5 (CCR5), angiotensin II receptor type 1 (AGTR1), solute carrier family 2 member 2 (facilitated glucose transporter SLC2A2), adiponectin (ADIPOQ), fatty acid binding protein 2 (FABP2), glutamine-fructose-6-phosphate transaminase 2 (GFPT2), advanced glycosylation end product-specific receptor (AGER), lymphotoxin alpha (LTA), vascular endothelial growth factor A (VEGF), ectonucleotide pyrophosphatase/phosphodiesterase 1 (ENPP1), SMT3 suppressor of mif two 3 homolog 4 (small ubiquitin-like modifier 4 protein SUMO4), estrogen receptor 1 (ESR1), superoxide dismutase 2 (SOD2), neuropeptide Y (NPY), engulfment and cell motility 1 (ELMO1), insulin-like growth factor binding protein 1 (IGFBP1), epidermal growth factor receptor (EGFR), paraoxonase 1 (PON1), aldo-keto reductase family 1, member B1 (AKRIB1), caldesmon 1 (CALD1), nitric oxide synthase 3 (NOS3), lipoprotein lipase (LPL), Pvt1 oncogene homolog MYC activator (PVT1), insulin (INS), xylosyltransferase I (XYLT), protein kinase C beta 1 (PRKCB1), solute carrier family 12 (SLC12A3), haptoglobin (HP), chemokine ligand 2 (CCL2), angiotensin I converting enzyme (ACE), meprin A, beta (MEP1B), carnosine dipeptidase 1 (CNDP1), intercellular adhesion molecule 1 (ICAM1), transforming growth factor beta 1 (TGFB1), apolipoprotein E (APOE) and superoxide dismutase 1 (SOD1), CD48 molecule (CD48), solute carrier family 35, member F3 (SLC35F3), endothelial PAS domain protein 1 (EPAS1), low density lipoprotein-related protein 1B (deleted in tumours) (LRP1B), dynein, axonemal, heavy chain 7 (DNAH7), ADAM metallopeptidase domain 23 (ADAM23), leprecan-likel (LEPREL1), human immunodeficiency virus type I enhancer binding protein 2 (HIVEP2), thrombospondin, type I, domain containing 7A (THSD7A), S-adenosylhomocysteine hydrolase-like 2 (AHCYL2), discs, large homolog 2 (Drosophila) (DLG2), FYVE, RhoGEF and PH domain containing 4 (FGD4), rabphilin 3A homolog (mouse) (RPH3A), citrate lyase beta like (CLYBL), protein kinase C, eta (PRKCH), epidermal growth factor receptor pathway substrate 15-like 1 (EPS15L1), cystatin 9 (testatin) (CST9), pericentrin (PCNT2), matrix metallopeptidase 9 (gelatinase B, 92 kDa gelatinase, 92 kDa type IV collagenase) (MMP9), apolipoprotein C-I (APOC1), cysteinyl-tRNA synthetase (CARS), chimerin (chimaerin) 2 (CHN2), neurocalcin delta (NCALD), ectonucleotide pyrophosphatase/phosphodiesterase 1 (ENPP1), major histocompatibility complex, class II, DR beta 1 (HLA-DRB1), interleukin 8 (IL8), pleckstrin homology domain containing, family H (with MyTH4 domain) member 2 (PLEKHH2), angiotensinogen (serpin peptidase inhibitor, Glade A, member 8) (AGT) and others, reviewed in Maeda, S: Genetics of diabetic nephropathy. Ther Adv Cardiovasc Dis 2, 363-71 (2008).

Genes and markers in type-2 diabetes and obesity are described in US patent application publication No. 2007-0292412 to Salonen et al.

Proteomic Studies

Few proteomic studies have been so far conducted with the aim of identifying biomarkers of predictive value for diabetic nephropathy. Otu et al. (Diabetes Care (2007) 30:638-543) (WO 2007/056587) performed a nested case-control study on Pima Indians that allowed for identification of a 12-peak proteomic signature that was validated on a small number of individuals. However, the study did not permit the identification of the proteins from which these peaks belong and replication in other populations is needed prior to concluding the broad applicability of these biomarkers.

Up to date, there have been dozens of genetic variants and genome regions associated or linked to myocardial infarction, both in T2D patients as well as in non-diabetics. These have been comprehensively reviewed recently (Yamada, Y, S Ichihara, T Nishida: Molecular genetics of myocardial infarction. Genomic Med 2, 7-22 (2008)) and specifically for genome-wide association studies in A Catalog of Published Genome-Wide Association Studies by The National Human Genome Research Institute (available on the world wide web at genome.gov/26525384) Johnson, A D, C J O'Donnell: An open access database of genome-wide association results. BMC Med Genet 10, 6 (2009).

### SUMMARY OF THE INVENTION

The present invention relates to previously unknown associations between T2D-related complications and various polymorphisms, genes and loci. These associated polymorphisms, genes, and loci provide basis for novel methods and kits for risk assessment, diagnosis and prognosis of T2D-related complication in a patient, among other things. In addition these polymorphisms, genes, and loci provide basis for methods and kits for novel therapies to prevent, treat and/or reduce risk of developing these complications.

A “biomarker” in the context of the present invention refers to a genetic feature such as, for example, single nucleotide polymorphism (SNP) or a short tandem repeat (STR). Other types of biomarkers include, but are not limited to, transcriptional products (such as, for example, mRNA or cDNA sequences thereof) or translational products (such as, for example, proteins or polypeptides) of genes comprising such SNPs. Representative examples of such SNPs are disclosed in Table 1, 4, 7, 10, 13, 14, 16 and 19. Polymorphic genes of the present invention comprise the genes/loci also disclosed in Table 3, 6, 9 and 12. A “biomarker” can also be a clinical or biological biomarker. Clinical or biological biomarkers include, but are not limited to, age, sex, glucose levels, age of diagnosis, diabetes duration at baseline, cigarette smoking, diastolic or systolic blood pressure, atrial fibrillation, glycated hemoglobin (HbA1_{c}), total cholesterol, HDL cholesterol, albumin/creatinine ratio, glomerular filtration rate.

In one embodiment, the biomarker is one of the SNPs listed in Table 1, 4, 7, 10, 13, 14, 16 and 19 or a SNP or a STR found to be in linkage disequilibrium to one of the SNP listed in Table 1, 4, 7, 10, 13, 14, 16 and 19.

Preferably, the biomarker is not a SNP listed in Table 20.

In another embodiment, the biomarker is a SNP of at least one of the genes listed in table 3, 6, 9 and 12 or a STR linked to a SNP of at least one of these above genes or to a locus closely related thereto.

Preferably, the biomarker is not an SNP of a gene which is listed in Table 21.

The present invention thus provides for methods of predicting risk of complications associated with T2D, comprising detecting at least one of the SNPs listed in Table 1, 4, 7, 10, 16 and 19 or a SNP or a STR found to be in linkage disequilibrium with one or more of the SNPs listed in Table 1, 4, 7, 10, 16 and 19, or a SNP of at least one gene listed in Table 3, 6, 9 and 12 or a SNP or a STR found to be in linkage disequilibrium with a SNP of such a gene, wherein the presence of the SNP or STR in a sample of a subject (or patient) suffering from T2D indicates that said subject (or patient) is likely to develop the complication. Preferred examples of such complications include, but are not limited to, albuminuria and/or declining glomerular filtration, myocardial infarction, and/or stroke.

As used herein, “single nucleotide polymorphism,” or “SNP” is a DNA sequence variation that occurs when a nucleotide, e.g., adenine (A), thymine (T), cytosine (C), or guanine (G), in the genome sequence is altered to another nucleotide. SNPs are occasional variations in DNA sequence; the vast majority of the DNA sequence is identical among all humans. SNPs or other variants may also be found in genomic regions that do not contain genes. They represent a genomic hot spot responsible for the genetic variability among humans.

As used herein, “gene” means any amount of nucleic acid material that is sufficient to encode a transcript or protein having the function desired. Thus, it includes, but is not limited to, genomic DNA, cDNA, RNA, and nucleic acid that are otherwise genetically engineered to achieve a desired level of expression under desired conditions. Accordingly, it includes fusion genes (encoding fusion proteins), intact genomic genes, and DNA sequences fused to heterologous promoters, operators, enhancers, and/or other transcription regulating sequences. Methods and nucleic acid constructs for preparing genes for recombinant expression are well known and widely used by those of skill in the art, and thus need not be detailed here. The term refers to an entirety containing entire transcribed region and all regulatory regions of a gene. The transcribed region of a gene including all exon and intron sequences of a gene including alternatively spliced exons and introns so the transcribed region of a gene contains in addition to polypeptide encoding region of a gene also regulatory and 5′ and 3′ untranslated regions present in transcribed RNA.

The genes of the invention are listed in Table 3, 6, 9 and 12.

Preferably, the gene is not one of the genes listed in Table 21.

As used herein, an “exon” is a segment of a eukaryotic gene that encodes a sequence of nucleotides in mRNA. An exon can encode amino acids in a protein. Exons are generally adjacent to introns.

As used herein, an “intron” is a non-coding region of a eukaryotic gene that may be transcribed into an RNA molecule, but is not usually translated into amino acids. It may be excised by RNA splicing when mRNA is produced.

As used herein, a “patient” is any living animal, including, but not limited to, a human who has, or is suspected of having or being susceptible to, a disease or disorder, or who otherwise would be a subject of investigation relevant to a disease or disorder. Accordingly, a patient can be an animal that has been bred or engineered as a model for metabolic syndrome, type 2 diabetes, obesity, hypertension, atherosclerosis, or any other disease or disorder. Likewise it can be a human suffering from, or at risk of developing, a disease or disorder associated with insulin metabolism, or any other disease or disorder. Similarly, a patient can be an animal (such as an experimental animal, a pet animal, a farm animal, a dairy animal, a ranch animal, or an animal cultivated for food or other commercial use), or a human, serving as a healthy control for investigations into diseases and/or disorders, e.g., those associated with insulin metabolism.

By “reagent,” is meant any element, molecule, or compound that is present in the assay system and participates, either directly or indirectly, in the biochemical processes occurring during the performance of the method. Reagents include, but are not limited to, nucleic acids, cells, media, chemicals, compounds used to introduce nucleic acids into cells, and compounds used to generate detectable signals.

By “materials” is meant items that are used to contain and/or perform the methods of the invention, but that do not participate in any of the biochemical reactions taking place in the method. Materials include, but are not limited to, test tubes, pipettes, gels, and ultraviolet transilluminators.

A “haplotype,” as described herein, refers to any combination of genetic markers (“alleles”) usually inherited together. A haplotype can comprise two or more alleles and the length of a genome region comprising a haplotype may vary from few hundred bases up to hundreds of kilobases. As it is recognized by those skilled in the art, the same haplotype can be described differently by determining the haplotype defining alleles from different nucleic acid strands. For example, the haplotype GGC defined by the SNP markers of this invention is the same as haplotype CCG in which the alleles are determined from the other strand, or haplotype CGC, in which the first allele is determined from the other strand. The haplotypes described herein are differentially present in T2D patients with increased risk of developing one or more of the aforementioned complications. Therefore, these haplotypes have diagnostic value for risk assessment, diagnosis and prognosis of T2D-related complications. Detection of haplotypes can be accomplished by methods known in the art used for detecting nucleotides at polymorphic sites.

A nucleotide position in genome at which more than one sequence is possible in a population, is referred to herein as a “polymorphic site” or “polymorphism”. Where a polymorphic site is a single nucleotide in length, the site is referred to as a SNP. For example, if at a particular chromosomal location, one member of a population has an adenine and another member of the population has a cytosine at the same position of his or her paternal or maternal DNA molecule, then this position is a polymorphic site, and, more specifically, the polymorphic site is a SNP. Polymorphic sites may be several nucleotides in length due to insertions, deletions, conversions or translocations. Each version of the sequence with respect to the polymorphic site is referred to herein as an “allele” of the polymorphic site. Thus, in the previous example, the SNP allows for both an adenine allele and a cytosine allele. Typically, a reference nucleotide sequence is referred to for a particular polymorphism e.g. in NCBI databases (as accessible on the world-wide-web at ncbi.nlm.nih.gov). Alleles that differ from the reference are referred to as “variant” alleles. The polypeptide encoded by the reference nucleotide sequence is the “reference” polypeptide with a particular reference amino acid sequence, and polypeptides encoded by variant alleles are referred to as “variant” polypeptides with variant amino acid sequences. Nucleotide sequence variants can result in changes affecting properties of a polypeptide. These sequence differences, when compared to a reference nucleotide sequence, include insertions, deletions, conversions and substitutions: e.g. an insertion, a deletion or a conversion may result in a frame shift generating an altered polypeptide; a substitution of at least one nucleotide may result in a premature stop codon, amino acid change or abnormal mRNA splicing; the deletion of several nucleotides, resulting in a deletion of one or more amino acids encoded by the nucleotides; the insertion of several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by the genes comprising such SNPs. For example, a nucleotide change resulting in a change in polypeptide sequence can alter the physiological properties of a polypeptide dramatically by resulting in altered activity, distribution and stability or otherwise affect on properties of a polypeptide. Alternatively, nucleotide sequence variants can result in changes affecting transcription of a gene or translation of its mRNA. A polymorphic site located in a regulatory region of a gene may result in altered transcription of a gene e.g. due to altered tissue specificity, altered transcription rate or altered response to transcription factors. A polymorphic site located in a region corresponding to the mRNA of a gene may result in altered translation of the mRNA e.g. by inducing stable secondary structures to the mRNA and affecting the stability of the mRNA. Such sequence changes may alter the expression of a susceptibility gene, such as, for example, an SNP associated with the aforementioned genes.

The SNP markers of the present invention, which are disclosed in tables 1, 4, 7, 10, 13, 14, 16 and 19 have been denoted with their official reference SNP (rs) ID identification tags assigned to each unique SNP by the National Center for Biotechnological Information (NCBI). Each rs ID has been linked to specific variable alleles present in a specific nucleotide position in the human genome, and the nucleotide position has been specified with the nucleotide sequences flanking each SNP.

Although the numerical chromosomal position of a SNP may still change upon annotating the current human genome build the SNP identification information such as variable alleles and flanking nucleotide sequences assigned to a SNP will remain the same. Those skilled in the art will readily recognize that the analysis of the nucleotides present in one or more SNPs set forth in table 1, 4, 7, 10, 13, 14, 16 and 19 of this invention in an individual's nucleic acid can be done by any method or technique capable of determining nucleotides present in a polymorphic site using the sequence information assigned in prior art to the rs IDs of the SNPs listed in table 1, 4, 7, 10, 13, 14, 16 and 19 of this invention. As it is obvious in the art the nucleotides present in polymorphisms can be determined from either nucleic acid strand or from both strands.

In one embodiment, the invention relates to a method for predicting the risk of developing a complication which is albuminuria and/or declining glomerular filtration myocardial infarction, or stroke in a subject having T2D, comprising detecting in a sample obtained from said subject at least one SNP having an RefSNP ID listed in Table 1, 4, 7, 10, 16 or 19.

In a related aspect, the present invention relates to a method for predicting the risk of developing a complication which is either myocardial infarction, or stroke or albuminuria and/or declining glomerular filtration or any combination thereof in a subject having T2D, comprising detecting at least one SNP having an RefSNP ID listed of in Table 1, 4, 7, 10, 16 or 19.

In a related aspect, the present invention relates to a method for predicting the risk of developing a complication which is albuminuria and/or declining glomerular filtration myocardial infarction, or stroke in a subject having T2D, comprising detecting in a sample obtained from said subject at least one SNP having an RefSNP ID listed in Table 1, 4, 7, 10, 16 or 19, wherein said RefSNP ID is not one of the SNPs listed in Table 20.

Determination of Repeated Sequences

The present invention also provides a method for prognosticating T2D-related complication in a subject comprising detecting short tandem repeats (STR) in linkage disequilibrium with a SNP listed in Table 1, 4, 7, 10, 16 or 19. The present invention thus provides for methods of predicting risk of complication associated with T2D, comprising detecting at least one STR found to be in linkage disequilibrium with one of the SNPs of the present invention, wherein the presence of the STR in a sample of a subject (or patient) suffering from T2D indicates that said subject (or patient) is likely to develop the complication. Preferred examples of such complications include, but are not limited to, albuminuria and/or declining glomerular filtration myocardial infarction, and/or stroke. Methods for determining the presence of repeated sequences in a nucleic acid sample (for example, genomic DNA) are known in the art.

As such, in a related embodiment, the present invention provides a method for prognosticating type 2 diabetes-related complication in a subject comprising detecting single tandem repeats (STR) in a nucleic acid target sequence, wherein such target sequences are contained in at least one gene from the aforementioned gene set or a locus related thereto. The nucleotide sequences contained in the genes and/or a locus related thereto are obtainable from the GENEID and/or OMIM accession numbers.

Geo-ethnic specificity. Given the density of the currently available genomic markers, it was possible to distinguish without any overlap Caucasians, Africans and Asians. However, the heterogeneity within Caucasians was not described prior to the initial filing date of the present patent application. Since then there has been several papers published describing the use of genetic variation for determination of geoethnic characteristics of an individual, e.g. Novembre, J, T Johnson, K Bryc et al.: Genes mirror geography within Europe. Nature 456, 98-101 (Nov. 6, 2008); Paschou, P, P Drineas, J Lewis et al.: Tracing sub-structure in the European American population with PCA-informative markers. PLoS Genet 4, e1000114 (Jul. 4, 2008); McEvoy, B P, G W Montgomery, A F McRae et al.: Geographical structure and differential natural selection among North European populations. Genome Res 19, 804-14 (2009). The present invention identified a composite of 14839 SNPs unrelated to complications (listed in Table 13), which allowed us to distinguish three sub-populations within Caucasians of European descent. Two of those are predominant with a west/east cline in Europe. The west type includes individuals from Ireland, Netherlands, United Kingdom but also a majority of people from Australia, Canada and New-Zealand (see FIG. 2). East type includes population from Russia, Estonia, Poland, Slovakia, Czech Republic and Hungary with German being populated by both geo-ethnic groups. In addition, Caucasians in Australia, Canada and New-Zealand include a significant admixture of the Eastern type. We have also identified a third group present as a minority in most of countries studied. Further refinement of the method allowed for identification of a composite of as few as 2 000 SNPs permitting the same (Table 14).

The geo-ethnic specificity has to be taken into account for the development of predictive tools based on genomic signatures which, as exemplified in FIG. 4, can result in higher/lower frequency of risk/protective alleles or difference in penetrance.

It is understood that the SNP markers of this invention may be associated with other polymorphisms. This allows for tagging SNPs (tagSNPs), which comprise loci that can serve as proxies for many other SNPs. The use of tagSNPs greatly improves the power of association studies as only a subset of loci needs to be genotyped while maintaining the same information and power as if one had genotyped a larger number of SNPs.

By using the name of the aforementioned gene provided in Table 3, 6, 9 and 12 those skilled in the art will readily find the nucleotide sequences of a gene and the mRNAs encoded thereby as well as amino acid sequences the encoded polypeptides.

In certain methods described herein, an individual who is at risk for a T2D-related complication is an individual in whom one or more SNPs selected from the Table 1, 4, 7, 10, 16 and 19 are identified. In other embodiment also polymorphisms or haplotypes associated to SNPs of the tables may be used in risk assessment of a T2D-related complication. The significance associated with an allele or a haplotype is measured by an odds ratio. In a further embodiment, the significance is measured by a percentage. In one embodiment, a significant risk is measured as odds ratio of 0.9 or less or at least about 1.1, including by not limited to: 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 10%, including but not limited to about 10%, 25%, 30%, 35%, 40%, 45%, 50%, 55%, 60%, 65%, 70%, 75%, 80%, 85%, 90%, 95% and 99%. In a further embodiment, a significant increase in risk is at least about 50%. It is understood however, that identifying whether a risk is medically significant may also depend on a variety of factors such as family history of hypertension, history of gestational diabetes, previously identified glucose intolerance, obesity, hypertriglyceridemia, hypercholesterolemia, elevated LDL cholesterol, low HDL cholesterol, elevated blood pressure (BP), cigarette smoking, lack of physical activity, and inflammatory components as reflected by increased C-reactive protein levels or other inflammatory markers.

“Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize to its specific target. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template may also include “non-specific priming sequences” or “nonspecific sequences” to which the primer or probe has varying degrees of complementarity. Probes and primers may include modified bases as in polypeptide nucleic acids. Probes or primers typically comprise about 15 to 30 consecutive nucleotides present e.g. in human genome and they may further comprise a detectable label, e.g., radioisotope, fluorescent compound, enzyme, or enzyme co-factor. Probes and primers to a SNP marker disclosed in table 1, 4, 7, 10, 16 and 19 are available in the art or can easily be designed using the flanking nucleotide sequences assigned to a SNP rs ID and standard probe and primer design tools.

Primers and probes for SNP markers disclosed in Table 1, 4, 7, 10, 16 and 19 can be used in risk assessment as well as molecular diagnostic methods and kits of this invention.

The invention comprises polyclonal and monoclonal antibodies that bind to a polypeptide encoded by a gene listed in table 3, 6, 9 or 12 or comprising a SNP set forth in Table 1, 4, 7, 10, 16 or 19 of the invention. The term “antibody” as used herein refers to immunoglobulin molecules or their immunologically active portions that specifically bind to an epitope (antigen, antigenic determinant) present in a polypeptide or a fragment thereof, but does not substantially bind other molecules in a sample, e.g., a biological sample, which contains the polypeptide. Examples of immunologically active portions of immunoglobulin molecules include F(ab) and F(ab′)₂ fragments which can be generated by treating the antibody with an enzyme such as pepsin. The term “monoclonal antibody” as used herein refers to a population of antibody molecules that are directed against a specific epitope and are produced either by a single clone of B cells or a single hybridoma cell line. Polyclonal and monoclonal antibodies can be prepared by various methods known in the art. Additionally, recombinant antibodies, such as chimeric and humanized monoclonal antibodies, comprising both human and non-human portions, can be produced by recombinant DNA techniques known in the art. Antibodies can be coupled to various enzymes, prosthetic groups, fluorescent materials, luminescent materials, bioluminescent materials, or radioactive materials to enhance detection.

In a related embodiment, the present invention also provides for the use of antisense oligonucleotides or silencing RNAs or similar methods which are capable of modulating the expression and/or levels of a product (i.e., mRNA or polypeptide) of a gene comprising a SNP set forth in Table 1, 4, 7, 10, 16 or 19. In a particularly preferred embodiment, the antisense molecules silencing RNAs or similar methods of the present invention are useful directed against the primary transcript (i.e., mRNA) of the genes listed in Table 3, 6, 9 and 12 or comprising a SNP set forth in Table 1, 4, 7, 10, 16 or 19. Techniques for the design and use of antisense molecules or silencing RNAs or similar methods, for example, in in vitro and/or in vivo applications, are known in the art.

“A T2D-related complication” in the context of this invention refers to glucose intolerance, insulin resistance, metabolic syndrome, obesity, a microvascular complication of T2D such as retinopathy, nephropathy or neuropathy, or a macrovascular complication such as coronary heart disease, cerebrovascular disease, congestive heart failure, claudication or other clinical manifestation of atherosclerosis or arteriosclerosis.

Preferred types of “T2D-related complications” include, but are not limited to, cardiovascular diseases, retinopathy, neuropathy, and/or nephropathy.

Particularly preferred “T2D-related complications” include, but are not limited to, myocardial infarction, stroke, albuminuria and/or declining glomerular filtration

An antibody specific for a polypeptide encoded by a gene identified in table 3, 6, 9 or 12 or containing a SNP listed in table 1, 4, 7, 10, 16 or 19 of the invention can be used to detect the polypeptide in a biological sample in order to evaluate the abundance and pattern of expression of the polypeptide. Antibodies can be used diagnostically to monitor protein levels in tissue such as blood as part of a test predicting the susceptibility to complications, such as, for example, myocardial infarction, stroke and/or albuminuria and/or declining glomerular filtration or as part of a clinical testing procedure, e.g., to, for example, determine the efficacy of a given treatment regimen. Highly purified antibodies (e.g. monoclonal humanized antibodies specific to a polypeptide encoded by an associated gene of the invention and/or polymorphic gene may be produced using GMP-compliant manufacturing processes known in the art. These “pharmaceutical grade” antibodies can be used in novel therapies modulating activity and/or function of a polypeptide encoded the associated gene(s) disclosed herein.

This invention provides information on genomic markers that can be used to develop methods, reagents and kits useful to predict diabetes complications. Development of such methods, reagents and kits relies on methods known to those skilled in the art, including without limitation allele specific PCR amplification or detection of such alleles, with or without prior amplification, with allele specific probes, and DNA sequencing. Information on genomic DNA sequences from which PCR primers, hybridization probes, and sequencing primers can designed can be found in public databases using the rs ID provided for each SNP in Table 1, 4, 7, 10, 13, 14, 16 and 19.

Diagnostic Methods and Test Kits

One major application of the current invention is diagnosing a susceptibility to T2D related complications. The risk assessment methods and test kits of this invention can be applied to any diabetic patient as a screening or predisposition test, although the methods and test kits are also be applied to prediabetic patients and other subjects, preferably those with high-risk individuals (who have e.g. family history of T2D, history of gestational diabetes, previous glucose intolerance, obesity or any combination of these). Diagnostic tests that define genetic factors contributing to T2D complications might be used together with or independent of the known clinical risk factors to define an individual's risk relative to the general population. Better means for identifying those individuals susceptible for a T2D-related complication should lead to better preventive and treatment regimens, including more aggressive management of the risk factors for a T2D-related complication such as obesity, lack of physical activity, hypercholesterolemia, elevated LDL cholesterol, low HDL cholesterol, elevated BP, cigarette smoking and inflammatory components as reflected by increased C-reactive protein levels or other inflammatory markers. Physicians may use the information on genetic risk factors to convince particular patients to adjust their life style e.g. to stop smoking, to reduce caloric intake or to increase exercise.

In one embodiment of the invention, diagnosis of a susceptibility to T2D related complication in a subject is made by detecting one or more SNP markers disclosed in table 1, 4, 7, 10, 16 and 19 of this invention in the subject's nucleic acid. The presence of assessed SNP markers or haplotypes in individual's genome indicates subject's increased risk for said T2D related complication. The invention also pertains to methods of diagnosing a susceptibility to said complication in an individual comprising detection of a haplotype in a genetic aspect that is more frequently present in an individual having a T2D complication (affected), compared to the frequency of its presence an individual not having a T2D complication (control), wherein the presence of the haplotype is indicative of a susceptibility to T2D-related complication. A haplotype may be associated with a reduced rather than increased risk of said complication, wherein the presence of the haplotype is indicative of a reduced risk of T2D-related complication.

In other embodiment of the invention, diagnosis of susceptibility to T2D-related complication, is done by detecting in the subject's nucleic acid one or more polymorphic sites which are in linkage disequilibrium with one or more SNP markers disclosed in table 1, 4, 7, 10, 16 and 19 of this invention. For a therapeutic purpose, the most useful polymorphic sites are those altering the biological activity of a polypeptide encoded by a T2D related complication gene set forth in Table 3, 6, 9 or 12. Examples of such functional polymorphisms include, but are not limited to frame shifts; premature stop codons, amino acid changing polymorphisms and polymorphisms inducing abnormal mRNA splicing. Nucleotide changes resulting in a change in polypeptide sequence in many cases alter the physiological properties of a polypeptide by resulting in altered activity, distribution and stability or otherwise affect on properties of a polypeptide. Other useful polymorphic sites are those affecting transcription of a gene set forth in Table 3, 6, 9 or 12 or comprising a SNP listed in table 1, 4, 7, 10, 16, or 19 or translation of its mRNA due to altered tissue specificity, due to altered transcription rate, due to altered response to physiological status, due to altered translation efficiency of the mRNA and due to altered stability of the mRNA. The presence of nucleotide sequence variants altering the polypeptide structure and/or expression in said associated genes in individual's nucleic acid is diagnostic for susceptibility to T2D-related complication but for a diagnostic purpose, the variant may also be included in uncharted areas of the genome.

In diagnostic assays determination of the nucleotides present in one or more SNP markers of this invention, as well as polymorphic sites associated therewith can be done by any method or technique which can accurately determine nucleotides present in a polymorphic site. Numerous suitable methods have been described in the art. These methods include, but are not limited to, hybridization assays, ligation assays, primer extension assays, enzymatic cleavage assays, chemical cleavage assays and any combinations of these assays. The assays may or may not include PCR, solid phase step, a microarray, modified oligonucleotides, labeled probes or labeled nucleotides and the assay may be multiplex or singleplex. As it is obvious in the art the nucleotides present in a polymorphic site can be determined from either nucleic acid strand or from both strands.

In another embodiment of the invention, a susceptibility to a T2D-related complication is assessed from transcription products of one or more associated genes. Qualitative or quantitative alterations in transcription products can be assessed by a variety of methods described in the art, including e.g. hybridization methods, enzymatic cleavage assays, RT-PCR assays and microarrays. A test sample from an individual is collected and the alterations in the transcription of associated genes are assessed from the RNA molecules present in the sample. Altered transcription is diagnostic for a susceptibility to a T2D-related complication.

In another embodiment of the invention, diagnosis of a susceptibility to T2D-related complication is made by examining expression, abundance, biological activities, structures and/or functions of polypeptides encoded by one of the gene disclosed in Table 3, 6, 9 or 12. A test sample from an individual is assessed for the presence of alterations in the expression, biological activities, structures and/or functions of the polypeptides, or for the presence of a particular polypeptide variant (e.g., an isoform) encoded by a gene disclosed in Table 3, 6, 9 or 12. An alteration can be, for example, quantitative (an alteration in the quantity of the expressed polypeptide, i.e., the amount of polypeptide produced) or qualitative (an alteration in the structure and/or function of a polypeptide encoded by the polymorphic genes could be measured. Alterations in expression, abundance, biological activity, structure and/or function of polypeptides encoded by such polymorphic genes can be determined by various methods known in the art e.g. by assays based on chromatography, spectroscopy, colorimetry, electrophoresis, isoelectric focusing, specific cleavage, immunologic techniques and measurement of biological activity as well as combinations of different assays. An “alteration” in the polypeptide expression or composition, as used herein, refers to an alteration in expression or composition in a test sample, as compared with the expression or composition in a control sample and an alteration can be assessed either directly from the polypeptide itself or its fragment or from substrates and reaction products of said polypeptide. A control sample is a sample that corresponds to the test sample (e.g., is from the same type of cells), and is from an individual who is not affected by a T2D complication. An alteration in the expression, abundance, biological activity, function or composition of a polypeptide encoded by a polymorphic gene of the invention in the test sample, as compared with the control sample, is indicative of a susceptibility to developing complications. In another embodiment, assessment of the splicing variant or isoform(s) of a polypeptide encoded by a polymorphic gene can be performed directly (e.g., by examining the polypeptide itself), or indirectly (e.g., by examining the mRNA encoding the polypeptide, such as through mRNA profiling).

Yet in another embodiment, a susceptibility to a T2D-related complication can be diagnosed by assessing the status and/or function of biological networks and/or metabolic pathways related to one or more polypeptides encoded by a T2D-related complication risk gene of this invention. Status and/or function of a biological network and/or a metabolic pathway can be assessed e.g. by measuring amount or composition of one or several polypeptides or metabolites belonging to the biological network and/or to the metabolic pathway from a biological sample taken from a subject. Risk to develop said complication is evaluated by comparing observed status and/or function of biological networks and or metabolic pathways of a subject to the status and/or function of biological networks and or metabolic pathways of healthy controls.

Another major application of the current invention is diagnosis of a molecular subtype of a type II diabetic patient. Molecular diagnosis methods and kits of this invention can be applied to a person having type II diabetes. In one preferred embodiment, molecular subtype of T2D in an individual is determined to provide information of the molecular etiology of T2D. When the molecular etiology is known, better diagnosis and prognosis of T2D can be made and efficient and safe therapy for treating T2D-related complications in an individual can be selected on the basis of this genetic subtype. For example, a drug that is likely to be effective, for example, a blood glucose lowering agent, can be selected without trial and error. Physicians may use the information on genetic risk factors with or without known clinical risk factors to convince particular patients to adjust their life style and manage T2D risk factors and select intensified preventive and curative interventions for them. In other embodiment, biomarker information obtained from methods and kits of the present invention are used to select human subjects for clinical trials testing anti-diabetic drugs. The kits provided for diagnosing a molecular subtype of T2D in an individual comprise wholly or in part protocol and reagents for detecting one or more biomarkers and interpretation software for data analysis and T2D molecular subtype assessment.

The diagnostic assays and kits of the invention may further comprise a step of combining non-genetic information with the biomarker data to make risk assessment, diagnosis or prognosis of a T2D-related complication. Useful non-genetic information comprises, without limitations, are age, gender, smoking status, physical activity, waist-to-hip circumference ratio (cm/cm), the subject family history of T2D or obesity, history of gestational diabetes, previously identified glucose intolerance, obesity, hypertriglyceridemia, low HDL cholesterol, HT and particularly elevated BP and/or status of being hypertensive. The detection method of the invention may also further comprise a step determining blood, serum or plasma glucose, total cholesterol, HDL cholesterol, LDL cholesterol, triglyceride, apolipoprotein B and AI, fibrinogen, ferritin, transferrin receptor, C-reactive protein, serum or plasma insulin concentration.

The score that predicts the probability of developing a T2D-related complication may be calculated using art-known procedures including but not limited to logistic regression, support vector machines and neural networks. The results from the further steps of the method as described below render possible a step of calculating the probability of developing such T2D-related complication using a logistic regression equation. Alternative statistical models include, but are not limited to, Cox's proportional hazards' model, other iterative models and neural networking models.

Diagnostic test kits (e.g. reagent kits) of this invention comprise reagents, materials and protocols for assessing one or more biomarkers, and instructions and software for comparing the biomarker data from a subject to biomarker data from healthy and diseased people to make risk assessment, diagnosis or prognosis of a T2D related complication and optimized therapeutic suggestions. Useful reagents and materials for kits include, but are not limited to PCR primers, hybridization probes and primers as described herein (e.g., labeled probes or primers), allele-specific oligonucleotides, reagents for genotyping SNP markers, reagents for detection of labeled molecules, restriction enzymes (e.g., for RFLP analysis), DNA polymerases, RNA polymerases, DNA ligases, marker enzymes, antibodies which bind to altered or to non-altered (native) a polypeptide, means for amplification of nucleic acids fragments from one or more SNPs selected from the table 1, 4, 7, 10, 13, 14, 16 or 19, means for analyzing the nucleic acid sequence of one or more T2D-complication related SNPs, or means for analyzing the sequence of one or more amino acid residues of polypeptides encoded by genes comprising such SNPs, etc. In one embodiment, a kit for diagnosing susceptibility to a T2D-related complication comprises primers and reagents for detecting the nucleotides present in one or more SNP markers selected from the table 1, 4, 7, 10, 16 or 19 in individual's nucleic acid.

Selection of Patients for Clinical Trials

Diabetes is very commonly associated with a significant risk of subsequent complications, such as cardiovascular diseases, stroke, macrovascular complications, and/or microvascular complications. Health Authorities, especially the US Food and Drug Administration (FDA), are concerned about recent reports of an increased rate of cardiovascular complications associated with the use of some anti-diabetic drugs. In order to protect the population, the FDA has requested very costly clinical studies to evaluate the cardiovascular risk of new diabetes drugs. For instance, in July 2008, the FDA convened a two-day meeting to discuss whether morbidity/mortality cardiovascular outcomes trials should be part of the approval process for pharmacological therapies developed for T2D. The resulting guidance was issued in December 2008 (Guidance for Industry: Diabetes Mellitus—Evaluating cardiovascular risk in new antidiabetic therapy to treat type 2 diabetes). In summary, the current standard for evaluation of efficacy have been strengthened by requiring further safety assessments, including long term (minimum 2 years) cardiovascular evaluation, based either on meta-analysis or an ad hoc clinical trial with sufficient “hard” cardiovascular outcomes in the control arm. In this document the FDA insists on recruiting patients with higher risk of cardiovascular events. Further guidelines are soon expected soon for drugs that have already been approved. The new requirements may, in turn, lead to the discontinuation of new drug development, and redirect available funding for diabetes research to other conditions.

One way to improve outcome is to properly stratify patients for risk of adverse side effects. The current methods for selecting patient populations for such studies are based on well-established risk factors, such as previous cardiovascular events, unfavorable lipid or blood pressure profile, increased CRP, etc. These approaches are partially effective since clinical trials would require upwards of 50 000 subjects if unselected T2D patients were randomized. The use of clinical and biological biomarker-based characterization has reduced the pool size to about 15,000. Nonetheless, this still remains one of the most costly (more than US $150M) and time consuming steps in drug development. An example is a trial launched in November 2008 by Merck, Inc. on the cardiovascular safety of sitagliptin (JANUVIA) (NCT 00790205) in T2D subjects. This trial required 14,000 patients, even though pre-existing cardiovascular disease was utilized as a criterion for inclusion in this study.

Correlations between genomic signatures as defined by SNPs and/or STRs combined with clinical/biological biomarkers, and cardiovascular complications in T2D patients provide a way to improve a researcher's ability to perform clinical outcome studies in a population. Using the methods and compositions in accordance with the embodiments of the instant invention herein disclosed, it is possible to reduce dramatically the number of subjects, and hence the cost of clinical studies, by selecting a filtered patient cohort comprising, for example, high-risk patients that are more likely to develop more cardiovascular complications than the general population of diabetes patients. Hence, in an embodiment of the instant invention, there is provided a means for identifying relevant genetic information and combining such information with other patient characteristics, such as, for example, age, sex, duration of diabetes, glycated hemoglobin, LDL and HDL cholesterol, hypertension, smoking, atrial fibrillation, ankle-arm blood pressure indices, pulse, symptomatic claudication and/or albuminuria, etc., which will be made available to companies developing new anti-diabetic drugs. Using the methods and compositions of the present invention, a researcher/clinician can identify a suitable patient cohort. Such may include, for example, patients likely to develop one or more of the aforementioned T2D related complications, etc.

As such, in an embodiment of the present invention, there is provided a novel, genomic based classification tool for characterizing patients with higher risk for T2D complications. The use of such a classification tool can dramatically reduce the sample size (and/or the time and cost) required to perform clinical safety outcome studies in T2D. Such outcome studies are typically utilized in the clinical trial setting, and can also be utilized in animal testing.

As used herein, the term “clinical trial” means any research study designed to collect clinical data on responses to a particular treatment, and includes but is not limited to phase I, phase II and phase III clinical trials. Standard methods are used to define the patient population and to enroll subjects. Preferably, the clinical trials of the hereinbefore described embodiment of the instant invention relate to T2D and complications thereof, such as, for example, cardiovascular death, myocardial infarction, stroke, albuminuria and/or declining glomerular filtration, and the like.

As illustrated in the Examples, a randomized clinical trial with two arms was designed to test the impact of novel antidiabetic medication on the rate of cardiovascular events in T2D patients. In one arm, patients receive the usual medication (control arm) whereas, in the other arm (treatment arm), patients receive the novel antidiabetic medication in addition to the usual medication. The number of samples used in both arms is such that a difference of 20% between the two arms' respective annual event rates will be detected with 80% power at a fixed significance level of 5%. A representative trial is planned for 5 years.

Using the methods and the compositions of an embodiment of the instant invention, it is now possible to distinguish, without any overlap, Caucasians from Africans and Asians among T2D diabetic subjects. Using subsets of SNPs in number varying from almost 150 000 to 2 000, the present inventors have determined two predominant populations among Caucasians recruited in ADVANCE along the west/east cline in Europe, with a majority of the Western type in Australia and Canada. An excess of complications that is observed in predominantly Eastern regions is mainly related with the frequency of some risk alleles compared to Western regions. Less frequently, the increased disease prevalence was found to be at least partially dependent on the allele penetrance, i.e., the population frequency is the same but the impact on the individual is more severe given its his/her genomic/environmental background. A first set of results has been obtained after genotyping (using Affymetrix Genome-Wide Human SNP Arrays 5.0 and 6.0) samples and analyzing data from nearly 2,000 T2D patients of Caucasian origin from 15 different countries. A case-control design was favored because it provides sufficient power to detect significant associations between phenotypes and alleles. Discriminative accuracy of best associated SNPs was determined by the area under a “Receiver Operating Characteristics” (ROC) curve (AUC), which is widely used to quantify the predictive performance of different classifiers: a value of 0.5 refers to a random classification whereas a value of 1.0 refers to a perfect classification.

An embodiment of the present invention also provides the use of training sets to fit models, highlighting variables which could possibly predict the outcome of interest. Under certain aspects, the testing set can used to assess the classification accuracy of the model on new known events. Two different models of classification were used: logistic regression and support vector machines. Logistic regression is a well known method which models the probability of a binary variable representing the outcome of interest (event vs. non-event) as a function of quantitative and/or categorical predictors. Support vector machine searches for optimal hyperplanes that separate two classes (here cases and controls) by maximizing the distance between the classes' closest points. The two methods gave similar predictive performance. As described in the Examples section, initial results on T2D complications show that the AUC increases to over 0.9 as the number of the best associated SNPs increases to 1000. Adding SNPs not associated to complications did not increase the predictive accuracy of the 3 methods. From the best associated SNPs in the entire dataset 95 independent SNPs maintaining 0.85 predictive value were identified. Using ADVANCE data, it was demonstrated for the first time that significant genomic determinants exists for vascular complications in diabetes. Furthermore, the micro-heterogeneity within the Caucasian population points to distinct conclusions on the impact of specific risk alleles, with, on one side, a significant potential for development of personalised medicine and, on the other side, the relevance for public health.

Illustrative results include the following ones:

Identification of over 1000 SNPs highly significantly associated (p<10⁻⁵) with T2D complications (kidney damage, myocardial infarction and stroke).

Identification of risk and protective alleles

Identification of genes and pathways associated with a specific or combined T2D complications

Identification of a set of SNPs pertinent to geo-ethnic sub-groups among Caucasians to be used for stratification correction

Determination that each most associated SNP alone has a low predictive value, while the combination of the 95 best SNPs reaches a predictive value of 0.85 for T2D complications.

In an embodiment of the present invention, there is provided a method for correlating a genetic feature with an increased or reduced risk of developing a complication associated with type-2 diabetes (T2D) and utilization of such information in the recruitment of subjects in clinical trials. Such complications include, but are not limited to, myocardial infarction, stroke, albuminuria and/or declining glomerular filtration or a combination thereof.

Preferably, under this embodiment, the complication is stroke, albuminuria and/or declining glomerular filtration.

The genetic feature is preferably a SNP or a STR, or a combination thereof.

Under this embodiment, it is particularly preferable to detect a genetic feature which is

(a) at least one single nucleotide polymorphism (SNP) listed in Table 1, 4, 7, 10, 16 or 19;
(b) at least one SNP which is in linkage disequilibrium with at least one SNP of (a); or
(c) at least one short tandem repeat (STR) that is in linkage disequilibrium with at least one SNP of (a).

According to an embodiment of the present invention, there is thus provided a method for characterizing a subject for inclusion or exclusion from a clinical trial, comprising detecting, in a sample obtained from said subject, the presence or absence of at least one genetic feature which is

(a) at least one single nucleotide polymorphism (SNP) listed in Table 1, 4, 7, 10, 16 or 19;
(b) at least one SNP which is in linkage disequilibrium with at least one SNP of (a); or
(c) at least one short tandem repeat (STR) that is in linkage disequilibrium with at least one SNP of (a).

Preferably, the methods of the hereinbefore described embodiment of the instant invention involve detection of one or more of the aforementioned genetic features using techniques that are known in the art, such as those disclosed in the Examples. The present invention can also be practiced by using a wide variety of techniques and reagents which are known in the art for detecting the absence of the aforementioned genetic features, for example, using probe sequences that detect wild-type nucleic acid sequences.

In a related embodiment, there is provided a method for characterizing a subject for inclusion or exclusion from a clinical trial, comprising detecting, in a sample obtained from said subject, the presence or absence of at least one genetic feature which is an SNP or a STR of at least one gene which is listed in Table 3, 6, 9.

As described in the Brief Description of the Tables section of this application, the genes of Table 3 relate to the SNPs of Table 1 via whole genome association. Herein, one or more of the SNPs listed in Table 1 are located either inside the gene (exon, intron, 5′UTR, 3′UTR) or very close to the gene of Table 3 (as defined by NCBI). Similarly, the genes of Table 6 relate to the myocardial infarction SNPs of Table 4. The relation between the SNPs and the genes is similar to that of Table 1 and Table 3. Lastly, the genes of Table 9 and 12 relate to the kidney-complication associated SNPs of Table 7 and 10, respectively. Herein, one or more of the SNPs listed in Table 7 or 10 are located either inside the gene (exon, intron, 5′UTR, 3′UTR) or very close to the gene of Table 9 or 12, respectively (as defined by NCBI).

In a more preferred embodiment, there is provided a method for selecting a patient for clinical trials comprising detecting in a biological sample of said patient, the presence or absence of at least one SNP listed in Table 1, 4, 7, 10, 16 or 19, said SNP being selected on the basis of its p value of association with a complication, allele frequency, or odds ratio.

Under a related embodiment of the present invention, there is provided a method for characterizing patients for clinical trials based on the detection of a combination of biomarkers.

In such methods, better characterization of the patient cohort can be achieved. Any combinations of SNP listed in tables 1, 4, 7, 10, 16 and 19 may be detected. Such combinations can be developed on the basis of, for example, level of association with a complication of interest and on the frequency of other genetic features. Such other genetic features, for example, risk or protective alleles in the population, etc. may be included. Several methods are known by those skilled in the art to select appropriate markers.

Under such embodiments of the instant invention wherein a combination of SNPs is detected, it is particularly preferable to employ a combination of biomarkers provided in tables 16 and 19.

Under such embodiments, any two, any four, any five, any ten, any twenty, or more of the SNPs listed in Tables 16 and/or 19 may be detected.

The compositions and methods of the present invention also provide methods for reducing the cost and time for anti-diabetic drug development by “enriching” the outcome trial pool with pre-selected patients that are at greater risk of T2D-related complications. To this end, the present application describes methods for calculating a Risk Index Score, which combines clinical/biological biomarkers with genomic markers with high predictive performance. Such risk scores allow identification of a population subset with a higher complication rate. The Risk Index Score, can be optionally integrated into a Clinical Research Tool, thus facilitating evaluation of efficacy/safety balance in T2D by improving the signal to noise ratio.

In a related embodiment, the present application relates to kits and combinations that allow for practicing one or more of the aforementioned methods.

Under this embodiment, there is provided combinations and kits for identifying a subject for clinical trial, wherein said subject is affected by type-2 diabetes (T2D) comprising in one or more packages
(a) an oligonucleotide that specifically hybridizes to a SNP having the RefSNP ID listed in Table 1, 4, 7, 10, 16 or 19; or
(b) an oligonucleotide which is the complement of (a);
and one or more reagents for the detection of said oligonucleotide.

Methods of Therapy

The present invention discloses novel methods for the prevention and treatment of a T2D-related complication. In particular, the invention relates to methods of treatment of T2D-related complications. The term, “treatment” as used herein, refers not only to ameliorating symptoms associated with the disease, but also preventing or delaying the onset of the complication, and also lessening the severity or frequency of symptoms of the disease, preventing or delaying the occurrence of a second episode of the disease or condition; and/or also lessening the severity or frequency of symptoms of the disease or condition.

The present invention encompasses methods of treatment (prophylactic and/or therapeutic) for a T2D-related complication using a therapeutic agent. A “therapeutic agent” is an agent that alters (e.g., enhances or inhibits) enzymatic activity or function of a risk gene such as those disclosed in Table 3, 6, 9 and 12 and/or expression of polymorphisms disclosed in table 1, 4, 7, 10, 16 and 19 and/or the specific metabolic or other biologically related pathway implicating those genes. The modes of useful therapeutic agents are further disclosed.

Representative therapeutic agents of the invention comprise the following: (a) nucleic acids, fragments, variants or derivatives of the genes, nucleic acids, or an active fragment or a derivative thereof and nucleic acids modifying the expression of said genes (e.g. antisense polynucleotides, catalytically active polynucleotides (e.g. ribozymes and DNAzymes), molecules inducing RNA interference (RNAi) and micro RNA), and vectors comprising said nucleic acids; (b) polypeptides, active fragments, variants or derivatives thereof, binding agents of polypeptides; peptidomimetics; fusion proteins or prodrugs thereof, antibodies; (c) metabolites of the gene products; (d) small molecules and compounds that alter (e.g., inhibit or antagonize) a risk gene expression, activity and/or function of a polypeptide encoded by said genes and; (e) small molecules and compounds that alter (e.g. induce, agonize or modulate) the expression or activity of said genes.

The nucleic acid sequences of T2D-related complication associated risk genes such as those disclosed in Table 3, 6, 9 and 12 and/or polymorphisms disclosed in table 1, 4, 7, 10, 16 and 19 of this application are publicly available and can be used to design and develop therapeutic nucleic acid molecules and recombinant DNA molecules for the prevention and treatment of T2D or a T2D related condition. For example antisense nucleic acid molecules targeted to a polymorphism in tables 1, 4, 7, 10, 16 and 19 can be designed using tools and the nucleotide sequence of the gene available in the art and constructed using chemical synthesis and/or enzymatic ligation reactions using procedures known in the art. Antisense nucleic acid molecule can be chemically synthesized using naturally occurring nucleotides or modified nucleotides designed to increase the biological stability of the molecules or to increase the physical stability of the duplex formed between the antisense oligonucleotide and sense nucleic acids, e.g., phosphorothioate derivatives and acridine substituted nucleotides can be used.

Alternatively, the antisense nucleic acid molecule can be produced biologically using an expression vector into which a nucleic acid molecule encoding a T2D-related complication risk gene, a fragment or a variant thereof has been cloned in antisense orientation (i.e., RNA transcribed from the expression vector will be complementary to the transcribed RNA of a T2D-related complication risk gene of interest).

More than one T2D-related complication therapeutic agent can be used concurrently, if desired. The therapy is designed to affect 1) expression of a T2D-related complication gene in a sense of activation, inhibition or modulation, 2) abundance, stability, biological activity and/or function of a T2D-related complication risk gene-encoded ribonucleic acid or polypeptide, or 3) biological activity and/or function of a T2D-related complication gene related signaling or metabolic pathway. Upregulation or increasing expression of a T2D-related complication risk gene or a particular variant of a T2D-related complication 4, 7, 10, 16 and 19 susceptibility gene could interfere with or compensate for the expression or activity of a defective gene or variant; downregulation or decreasing expression or availability of a native risk gene or a particular splicing variant of a T2D-related complication susceptibility gene could minimize the expression or activity of a defective gene or the particular variant and thereby minimize the impact of the defective gene or the particular variant.

The T2D and T2D-related complication therapeutic agent(s) are administered in a therapeutically effective amount that can be determined using established clinical methods and assays. The precise dose to be employed in the formulation will also depend on the route of administration, and the seriousness of the disease or disorder, and should be decided according to the judgment of a practitioner. Effective doses may be extrapolated from dose-response curves derived from in vitro or animal model test systems.

In one embodiment, a nucleic acid encoding a T2D-related complication polypeptide, fragment, variant or derivative thereof, either by itself or included within a vector, can be introduced into cells of an individual affected by T2D or a T2D related condition using variety of experimental methods described in the art, so that the treated cells start to produce native T2D-related complication susceptibility polypeptide. Thus, cells which, in nature, lack of a native T2D-related complication risk gene expression and activity, or have abnormal T2D-related complication risk gene expression and activity, can be engineered to express a T2D-related complication polypeptide or an active fragment or a different variant of said T2D-related complication susceptibility polypeptide. Genetic engineering of cells may be done either “ex vivo” (i.e. suitable cells are isolated and purified from a patient and re-infused back to the patient after genetic engineering) or “in vivo” (i.e. genetic engineering is done directly to a tissue of a patient using a vehicle). Alternatively, in another embodiment of the invention, a nucleic acid (e.g. a polynucleotide) which specifically hybridizes to the mRNA and/or genomic DNA of a T2D-related complication gene is administered in a pharmaceutical composition to the target cells or said nucleic acid is generated “in vivo”. The antisense nucleic acid that specifically hybridizes to the mRNA and/or DNA inhibits expression of the T2D-related complication polypeptide, e.g., by inhibiting translation and/or transcription. Binding of the antisense nucleic acid can be due to conventional base pairing, or, for example, in the case of binding to DNA duplexes, through specific interaction in the major groove of the double helix. In a preferred embodiment nucleic acid therapeutic agents of the invention are delivered into cells that express one or more T2D-related complication risk genes. A number of methods including, but not limited to, the methods known in the art can be used for delivering a nucleic acid to said cells. For example, a vector can be introduced in vivo such that it is taken up by a cell and directs the transcription of a RNA molecule, which induces RNA interference in the cell. Such a vector can remain episomal or become chromosomally integrated, and as long as it can be transcribed to produce the desired RNA molecules it will modify the expression of a T2D-related complication risk gene. Such vectors can be constructed by various recombinant DNA technology methods standard in the art.

The expression of an endogenous T2D-related complication risk gene can be reduced by inactivating completely (“knocking out”) or partially (“knocking down”) a T2D-related complication gene using targeted homologous recombination methods described in the art. Alternatively, expression of a functional, non-mutant T2D-related complication can be increased using a similar method: targeted homologous recombination can be used to replace a non-functional T2D-related complication risk gene with a functional form of the said gene in a cell. In another embodiment of the invention, other T2D therapeutic agents as described herein can also be used in the treatment or prevention of T2D or a related condition. The therapeutic agents can be delivered in a pharmaceutical composition; they can be administered systemically, or can be targeted to a particular tissue. The therapeutic agents can be produced by a variety of means, including chemical synthesis, cell culture and recombinant techniques (e.g. with transgenic cells and animals). Therapeutic agents can be isolated and purified to fulfill pharmaceutical requirements using standard methods described in the art. A combination of any of the above methods of treatment (e.g., administration of non-mutant T2D-related complication susceptibility polypeptide in conjunction with RNA molecules inducing RNA interference targeted to the mutant T2D-related complication susceptibility mRNA) can also be used.

In the case of pharmaceutical therapy the invention comprises compounds which affect 1) expression of a T2D-related complication gene in a sense of activation, inhibition or modulation, 2) abundance, stability, biological activity and/or function of a T2D-related complication risk gene-encoded ribonucleic acid or polypeptide, or 3) biological activity and/or function of a T2D-related complication gene or metabolic pathway encoded by T2D complication-associated risk genes such as those disclosed in Table 3, 6, 9 and 12 and/or polymorphisms disclosed in table 1, 4, 7, 10, 16 and 19 of this application. The treatment may also exert its effects as specified above on one or several genes selected from the T2D complication-associated risk genes such as those disclosed in Table 3, 6, 9 and 12 and/or polymorphisms disclosed in table 1, 4, 7, 10, 16 and 19 of this application.

Furthermore, a disclosed method or a test based on biomarkers specific for T2D-related complication susceptibility gene is useful in selection, modification or optimization of therapeutic modalities for T2D-patients. For example if the less frequent, i.e. the minor, assumable mutated allele in the T2D-related complication susceptibility gene is risk-reducing, and if said mutation is a gene function reducing mutation, one can deduce that the gene function and/or activity would increase the risk of T2D complication. On that basis, drugs and other therapies such as gene therapies that reduce or inhibit the function or activity of the T2D-related complication susceptibility gene or the encoded protein would reduce the risk of the said T2D-related complication and could be used to both prevent and treat the said T2D-related complication in subjects having said mutated allele.

In another embodiment of the invention a T2D or T2D-related complication therapeutic agent comprises a known therapeutic agent related to a T2D-related complication associated gene listed in tables 3, 6, 9 or 12 of this invention but which is not used to treat T2D or a T2D-related complication. Such compounds and therapeutic agents are useful for developing new therapies for T2D or a T2D-related complication as they most likely affect 1) expression of a T2D-related complication gene in a sense of activation, inhibition or modulation, 2) abundance, stability, biological activity and/or function of a T2D-related complication risk gene-encoded ribonucleic acid or polypeptide, or 3) biological activity and/or function of a T2D-related complication gene related signaling or metabolic pathway. These agents may be used alone or in combination with other treatments and agents used for prevention or treatment of T2D or a T2D-related condition.

In one embodiment of the invention, therapeutic agents or compounds currently utilized for the treatment of T2D and T2D-related complications are combined with one or more known therapeutic agents used to treat T2D comprising I. oral antidiabetics including biguanid derivatives such as 1) metformin, 2) buformin, insulin secretagogues such as 1) sulphonylurea derivatives such as tolbutamide, glibenclamide, gliclazide, glipizide, glimepiride, gliquidone; 2) meglitinides such as repaglinide, nateglinide; 3) alpha-glucosidase inhibitors such as acarbose, miglitol; 4) thiazolidinediones such as rosiglitazone and pioglitazone; 5) other defined by World Health Organisation—The Anatomical Therapeutic Chemical (ATC) classification system; II. insulin such as i) insulin glargine, ii) insulin aspart, iii) insulin lispro, iv) insulin glulisine; v) insulin detemir; and agents known do decrease and or prevent diabetes related complication, such as high blood pressure, i) converting enzyme inhibitors, ii) angiotensin receptor blockers, iii) direct renin inhibitors, iv) endothelin antagonists, v) diuretics, vi) beta blockers, vii) alpha blockers, viii) inhibitors of phosphodiesterase 5a and the combinations thereof.

Pharmaceutical Compositions

The present invention also pertains to pharmaceutical compositions comprising agents described herein, particularly polynucleotides, polypeptides and any fractions, variants or derivatives of T2D-related complication genes, and/or agents that alter (e.g., enhance or inhibit) expression of a risk gene or genes, or activity of one or more polypeptides encoded by associated genes as described herein. For instance, an agent that alters expression of a risk gene or activity of one or more polypeptides encoded thereby.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrolidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents. The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration. For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of a T2D-related complication, and should be decided according to the judgment of a practitioner and each patient's circumstances. Effective doses may be extrapolated from dose-response curves derived from in vitro or animal model test systems.

Functional Foods

By definition “functional foods” or “nutraceuticals” are foods or dietary components or food ingredients that may provide a health benefit beyond basic nutrition. Functional foods are regulated by authorities (e.g. by the FDA in US) according to their intended use and the nature of claims made on the package. Functional foods can be produced by various methods and processes known in the art including, but not limited to synthesis (chemical or microbial), extraction from a biological material, mixing functional ingredient or component to a regular food product, fermentation or using a biotechnological process. A functional food may exert its effects directly in the human body or it may function e.g. through human intestinal bacterial flora.

The associated genes disclosed in Table 3, 6, 9 and 12 of this invention can be used as molecular targets towards which functional foods claiming health benefit in a T2D related complication can be developed. For example a functional food may compensate reduced biological activity of a polypeptide encoded by a gene set forth in Table 3, 6, 9 or 12 when the risk gene is defective or is not expressed properly in a subject. A functional food may also inhibit the expression and/or biological activity of a gene or polypeptide of the invention promoting the development of a T2D related complication. In another embodiment a functional food may increase the expression and/or biological activity of a gene or polypeptide protecting an individual from the development of a T2D related complication due to reduced expression and protein production.

Aspects of the instant invention include, but are not limited to:

Aspect 1. A method for predicting a risk of developing a complication which is myocardial infarction, stroke, or albuminuria and/or declining glomerular filtration in a subject affected by T2D, comprising detecting, in a sample obtained from said subject, at least one genetic feature which is single nucleotide polymorphism (SNP), short tandem repeat (STR),

wherein the detection of said genetic feature in said subject correlates with said risk of developing at least one of said complication.

Aspect 2. The method according to aspect 1, wherein said SNP comprises a polymorphism of a gene or a locus linked thereto.
Aspect 3. The method according to aspect 1, wherein said genetic feature comprises at least one feature listed in Table 1, 4, 7, 10, 16 or 19.
Aspect 4. The method according to aspect 1, wherein said genetic feature is at least one SNP listed in Table 1, 4, 7, 10, 16 or 19.
Aspect 5. The method according to aspect 1, wherein said genetic feature is at least one SNP or one STR found to be in linkage disequilibrium with a SNP listed in Table 1, 4, 7, 10, 16 or 19.
Aspect 6. The method according to aspect 1, comprising detecting a SNP or a STR of at least one gene which is listed in Table 3, 6, 9 or 12.
Aspect 7. The method according to aspect 1, wherein detection of said genetic feature correlates with increased or reduced risk of developing said complication.
Aspect 8. A method for predicting a risk of developing a complication which is myocardial infarction, stroke, or albuminuria and/or declining glomerular filtration in a subject affected by type II diabetes, comprising detecting, in a sample obtained from said subject, at least one single nucleotide polymorphism (SNP) from the SNPs listed in Table 1, 4, 7, 10, 16 or 19, or at least one SNP or one TR found to be in linkage disequilibrium with at least one SNP listed in Table 1, 4, 7, 10, 16 or 19, wherein the detection of said SNP or STR in said subject correlates with said risk of developing said complication.
Aspect 9. A method for predicting a risk of developing a complication which is myocardial infarction, stroke, or albuminuria and/or declining glomerular filtration in a subject affected by T2D, comprising detecting, in a sample obtained from said subject, at least one (SNP) from at least on gene listed in Table 3, 6, 9 or 12 or at least one STR found to be in linkage disequilibrium with at least one (SNP) from at least one gene listed in Table 3, 6, 9 or 12, wherein the detection of said SNP or STR in said subject correlates with said risk of developing said complication.
Aspect 10: A method for predicting the risk of developing a complication which is myocardial infarction, stroke, albuminuria and/or declining glomerular filtration or any combination thereof in a subject having type II diabetes, comprising detecting at least one SNP from the list of SNPs of Table 1, 4, 7, 10, 16 or 19, said SNP being selected on the basis of its p value of association with said complication(s), allele frequency and odds ratio.
Aspect 11. The method according to aspects 8 or 9 or 10, comprising detecting at least two SNPs.
Aspect 12. The method according to aspects 8 or 9 or 10, comprising detecting at least three SNPs.
Aspect 13. The method according to aspects 8 or 9 or 10, comprising detecting more than three SNPs
Aspect 14. The method according to aspects 8 or 9 or 10, wherein said STR and/or SNP is detected in said patient in a specific geo-ethnic context.
Aspect 15: The method of aspect 14, wherein the geoethnic origin of an individual is determined using SNPs selected from the SNPs listed in Table 13 or 14.
Aspect 16. The method according to aspect 1, wherein the genetic feature is
(a) at least one single nucleotide polymorphism (SNP) listed in Table 16 or 19;
(b) at least one SNP which is in linkage disequilibrium with at least one SNP of (a); or
(c) short tandem repeat (STR) that is in linkage disequilibrium with at least one SNP of (a).
Aspect 17. The method according to Aspect 16, comprising detecting at least two SNPs from the SNPs listed in Table 16 or 19.
Aspect 18. The method according to Aspect 16, comprising detecting at least three SNPs from the SNPs listed in Table 16 or 19.
Aspect 19. The method according to aspect 1, with the proviso that said at least one SNP is not one of the SNPs listed in Table 20.
Aspect 20. The method according to aspect 2, with the proviso that said at least one gene is not one of the genes listed in Table 21.
Aspect 21. A kit for predicting a complication which is myocardial infarction, stroke or albuminuria in a subject affected by type-2 diabetes (T2D) comprising in one or more packages
(a) an oligonucleotide that specifically hybridizes to a SNP having the RefSNP ID listed in Table 1, 4, 7, 10, 16 or 19; or
(b) an oligonucleotide which is the complement of (a);
and one or more reagents for the detection of said oligonucleotide.
   Aspect 22. The kit according to aspect 21, comprising one or more reagents for polymerase chain reaction (PCR).
   Aspect 23. The kit of aspect 21, further comprising an RNase.
   Aspect 24. The kit of aspect 21, which further comprises one or more reagents for isolation of cells from a sample.
   Aspect 25. The kit of aspect 24, wherein said sample is a blood sample.
   Aspect 26. The kit of aspect 21, further comprising a DNAse inhibitor.
   Aspect 27. The kit of aspect 21, further comprising reagents for sequencing in one or more packages.
   Aspect 28. The kit of aspect 21, further comprising a cDNA microarray plate in one or more packages.
   Aspect 29. The kit of aspect 21, wherein the oligonucleotide comprises the probe sequences of Tables 2, 5, 8, 9, 11 and 16.
   Aspect 30. The kit of aspect 21, wherein the oligonucleotide is
(a) an oligonucleotide comprising at least 80% sequence identity to the oligonucleotide having the RefSNP ID listed in Table 1, 4, 7, 10, 16 or 19; or
(b) an oligonucleotide which is complimentary to the oligonucleotide of (a).
   Aspect 31. The kit of aspect 30, wherein the oligonucleotide is
(c) an oligonucleotide comprising at least 90% sequence identity to the oligonucleotide having the RefSNP ID listed in Table 1, 4, 7, 10, 16 or 19; or
(d) an oligonucleotide which is complimentary to the oligonucleotide of (a).
   Aspect 32. The kit of aspect 30, wherein the oligonucleotide is
(e) an oligonucleotide comprising at least 95% sequence identity to the oligonucleotide having the RefSNP ID listed in Table 1, 4, 7, 10, 16 or 19; or
(f) an oligonucleotide which is complimentary to the oligonucleotide of (a).
   Aspect 33. The kit of aspect 30, wherein the oligonucleotide is
(g) an oligonucleotide comprising at least 100% sequence identity to the oligonucleotide having the RefSNP ID listed in Table 1, 4, 7, 10, 16 or 19; or
(h) an oligonucleotide which is complimentary to the oligonucleotide of (a).
   Aspect 34. The kit of aspect 21, which further comprises a control oligonucleotide that hybridizes to the wild-type allele.
   Aspect 35. Use of a kit according to Aspect 21 for predicting a complication which is myocardial infarction, stroke, albuminuria or declining glomerular filtration in a subject affected by type-2 diabetes (T2D).
   Aspect 36. A method for preventing, treating or reducing the risk of T2D or a T2D related complication which is myocardial infarction, stroke, albuminuria or declining glomerular filtration in a subject in need thereof, comprising administrating to said subject a therapeutic agent which
(a) alters the enzymatic activity or levels of a polypeptide encoded by at least one risk gene listed in Table 3, 6, 9; or
(b) alters the expression of at least one polymorphism disclosed in table 1, 4, 7, 10, 16, or 19; or
(c) alters the specific metabolic or other biologically related pathway implicating the risk gene of (a).
   Aspect 37. The method according to aspect 36, wherein the therapeutic agent alters the expression of at least one polymorphism disclosed in table 1, 4, 7, 10, 16, or 19.
   Aspect 38. The method according to aspect 37, wherein the therapeutic agent is an antisense oligonucleotide or an siRNA.
   Aspect 39. The method according to aspect 36, wherein the therapeutic agent alters the levels or enzymatic activity of a polypeptide encoded by at least one risk gene listed in Table 3, 6, 9.
   Aspect 40. The method according to aspect 39, wherein the therapeutic agent is an antibody.
   Aspect 41. A method for identifying a subject for preventive therapeutic action, comprising detecting, in a sample obtained from said subject, at least one genetic feature which is
   (a) at least one single nucleotide polymorphism (SNP) listed in Table 1, 4, 7, 10, 16 or 19;
   (b) at least one SNP which is in linkage disequilibrium with at least one SNP of (a); or
   (c) short tandem repeat (STR) that is in linkage disequilibrium with at least one SNP of (a), wherein the detection of said genetic feature in said subject correlates with the eligibility of said subject for said preventive therapeutic action.

Additional aspects of the instant invention include, but are not limited to:

Aspect A. A method for characterizing a subject for inclusion or exclusion from a clinical trial, comprising detecting, in a sample obtained from said subject, the presence or absence of at least one genetic feature which is
(a) at least one single nucleotide polymorphism (SNP) listed in Table 1, 4, 7, 10, 16 or 19;
(b) at least one SNP which is in linkage disequilibrium with at least one SNP of (a); or
(c) at least one short tandem repeat (STR) that is in linkage disequilibrium with at least one SNP of (a).
Aspect B. The method according to Aspect A, comprising detecting a SNP or a STR of at least one gene which is listed in Table 3, 6, 9.
Aspect C. The method according to Aspect A, wherein detection of said genetic feature correlates with an increased or reduced risk of developing a complication associated with type 2 diabetes (T2D).
Aspect D. The method according to Aspect C, wherein detection of said genetic feature correlates with increased risk of developing said complication associated with T2D.
Aspect E. The method according to Aspect C, wherein said complication associated with T2D is myocardial infarction, stroke, albuminuria or declining glomerular filtration or a combination thereof.
Aspect F: The method according to Aspect E, comprising detecting at least one SNP from the list of SNPs of Table 1, 4, 7 or 10, said SNP being selected on the basis of its p value of association with said complication(s), allele frequency or odds ratio.
Aspect G. The method according to Aspect A, comprising detecting at least two SNPs.
Aspect H. The method according to Aspect A, comprising detecting at least three SNPs.
Aspect I. The method according to Aspect A, comprising detecting more than three SNPs.
Aspect J. The method according to Aspect A, wherein said STR and/or SNP is detected in said patient in a specific demographically-defined population.
Aspect K. The method according to Aspect A, wherein if said genetic feature is detected in said subject, then the subject is included in said clinical trial.
Aspect L. The method according to Aspect A, for characterizing a subject for inclusion in a clinical trial comprising detecting the presence of said at least one genetic feature.
Aspect M. The method according to Aspect A, for characterizing a subject for exclusion from a clinical trial comprising detecting the absence of said at least one genetic feature.
Aspect N. The method according to Aspect A, wherein the genetic feature is
(a) at least one single nucleotide polymorphism (SNP) listed in Table 16 or 19;
(b) at least one SNP which is in linkage disequilibrium with at least one SNP of (a); or
(c) short tandem repeat (STR) that is in linkage disequilibrium with at least one SNP of (a).
Aspect O. The method according to Aspect N, comprising detecting at least two SNPs from the SNPs listed in Table 16 or 19.
Aspect P. The method according to Aspect N, comprising detecting at least three SNPs from the SNPs listed in Table 16 or 19.
Aspect Q. The method according to aspect A, with the proviso that said at least one SNP is not one of the SNPs listed in Table 20.
Aspect R. The method according to aspect B, with the proviso that said at least one gene is not one of the genes listed in Table 21.

The entire disclosures of all applications, patents and publications, cited above and below, are hereby incorporated by reference.

### BRIEF DESCRIPTION OF THE DRAWINGS AND TABLES

Various features and attendant advantages of the present invention will be more fully appreciated as the same becomes better understood when considered in conjunction with the accompanying drawings, in which like reference characters designate the same or similar parts throughout the several views, and wherein:

FIG. 1. Genetic axis of variations were constructed using two principal components based on 14961 SNPs characterized by minor allele frequency of more than 10%, with a call rate of 100%, and HWE p>0.0001 equally distributed over the genome at every 100 kb, unrelated to complications on all autosomes.

FIG. 2. Histogram of number of individuals by the three main regions defined by PCA analysis described in FIG. 1, for each country included in the dataset.

FIG. 3. Prevalence of T2D complications (albuminuria and/or declining glomerular filtration, stroke and MI) in each country included in the dataset.

FIG. 4 shows three-panel, Upper, prevalence of the complication, Middle, frequency of the risk allele and lower, odds ratio of risk for all individuals or individual belonging to regions 1, 2 or 3 respectively, for selected examples of SNPs and complications. The allelic odds ratio (OR) were computed for the same phenotype as the association was found. The five examples shown are for A: SNP rs4664386, albuminuria, B: SNP rs 7557611, complication, C: SNP rs1516093, MI, D: SNP rs7126809, stroke and E, rs9457730, albuminuria. Panels A-C show that the frequency of the risk allele is higher in region 3 which also exhibits higher prevalence of the complications while panels D and E show same frequency of the risk allele for both regions but higher odds ratio of risk in region 1 or 3. The geo-ethnic specificity has to be taken into account for the development of predictive tools based on genomic signatures.

FIG. 5: AUC scores of best associated SNPs (with imputation) and random SNPs with leave-one-out cross-validation vs. number of SNPs. In order to use all of our subjects, genotype imputation was performed with fastPHASE. The missing genotype rate par person doesn't exceed 10%.

FIG. 6: AUC scores of 4 different datasets with leave-one-out cross-validation from 50 to 250 SNPs. Set 1: Imputed genotypes. Set 2: Imputed genotypes plus random SNPs 50*└N/2┘ random SNPs, N=[1,20]. Set 3: Random SNPs. Set 4: 95 SNPs after feature selection

FIG. 7: Results of bootstrap analysis using the four models described in Example 2 (Logistic regression).

FIG. 8: Impact of the annual event rate in the control arm on the number of patients which need to be enrolled at entry of a clinical trial aimed at detecting differences between cardiovascular events in T2D patients treated or not with new medication.

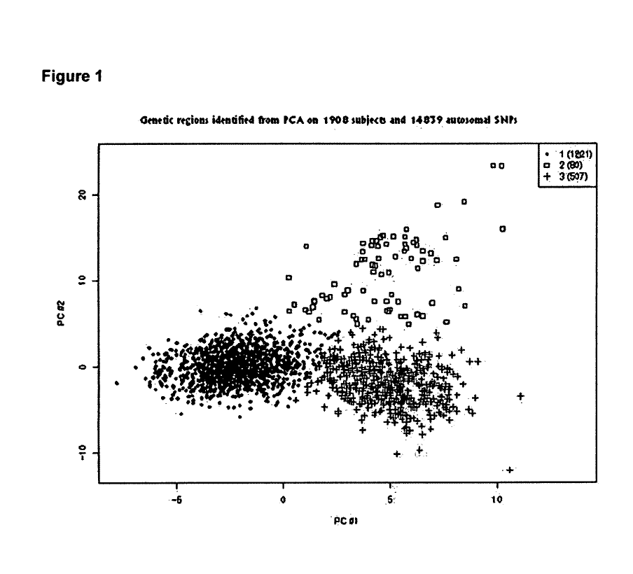

Table 1: Results of whole genome association (WGA) tests for three different sample sets: all 1908 individuals and individuals belonging to region 1 and region 3 respectively. WGA tests for three different samples given the population named 1908 individuals: all individuals with and without covariate (methods=pca1 (see FIG. 1) and all respectively), and those belonging to regions 1 (method=region-1) and 3 (method=region-3). The table contains the results of WGA tests on the following phenotypes: albuminuria, myocardial infarction, stroke, and all complications combined. SNPs selected have p-value<5×10e−04. Quality filters: minor allele frequency, MAF 0.01, HWE 0.001, call rate 0.98. Determination of the risk allele for OR: If the minor allele frequency is higher in cases than in controls, the minor allele is defined as the risk allele.

Table 2: Flanking sequences of each of the SNPs listed in Table 1.

Table 3. List of all genes relevant to the whole genome association results of Table 1. The relevance here means that one on more of the SNPs listed in Table 1 are located either inside the gene (exon, intron, 5′UTR, 3′UTR) or very close to the gene—as defined by NCBI.

Table 4. Myocardial infarction SNPs. In the first column (RS_ID), the official name of SNP according to dbSNP of NCBI is given; the second column (MARSHFILED) denotes the position of SNP in cM on the respective chromosome. METHOD encompasses the association model type (linear or logistic regression) and covariates used in the model (HBP . . . hypertension, GE . . . geoethnic structure as determined by principal components 1 and two of PCA analysis, ACR . . . albumin/creatinine ratio, CRCL . . . creatinine clearance, AGE and SEX). PHENOTYPE denotes the case and control setup as described above. CHR identifies chromosomal allocation of the SNPs. P-VALUE shows the p value and OR_ALL denotes allelic odds ratio

Table 5. Flanking sequences of each of the SNPs in Table 4. The table comprises the official name of SNP according to dbSNP of NCBI (dbSNP RS ID) and the flanking sequence as retrieved from queries to Affymetrix NetAffx™ Analysis Center for the SNPs included in Table 4.

Table 6. Genes related to MI SNPs from Table 4. The table shows the official symbol (GENE_SYMBOL) and name of the genes (Gene Name) according to Human Genome Organisation Gene Nomenclature Committee for the genes relevant to the whole genome association results of 1908 individuals in Table 4. The relevance here means that one on more of the SNPs listed in Table 4 are located either inside the gene (exon, intron, 5′UTR, 3′UTR) or very close to the gene—as defined by NCBI.

Table 7. Kidney related complication (qualitative phenotypes) SNPs. In the first column (RS_ID), the official name of SNP according to dbSNP of NCBI is given; the second column (MARSHFILED) denotes the position of SNP in cM on the respective chromosome. METHOD encompasses the association model type (linear or logistic regression) and covariates used in the model (HBP . . . hypertension, GE . . . geoethnic structure as determined by principal components 1 and two of PCA analysis, AGE and SEX). PHENOTYPE denotes the case and control setup as described above. CHR identifies chromosomal allocation of the SNPs. P-VALUE shows the p value and OR_ALL denotes allelic odds ratio.

Table 8. Flanking sequences of each of the SNPs in Table 7. The table comprises the official name of SNP according to dbSNP of NCBI (dbSNP RS ID) and the flanking sequence as retrieved from queries to Affymetrix NetAffx™ Analysis Center for the SNPs included in Table 7.

Table 9. Genes related to kidney SNPs from Table 7. The table shows the official symbol (GENE_SYMBOL) and name of the genes (Gene Name) according to Human Genome Organisation Gene Nomenclature Committee for the genes relevant to the whole genome association results of 1908 individuals in Table 7. The relevance here means that one on more of the SNPs listed in Table 7 are located either inside the gene (exon, intron, 5′UTR, 3′UTR) or very close to the gene—as defined by NCBI.

Table 10. Kidney related complication (quantitative phenotypes) SNPs. In the first column (RS_ID), the official name of SNP according to dbSNP of NCBI is given; the second column (MARSHFIELD) denotes the position of SNP in cM on the respective chromosome. METHOD encompasses the association model type (linear or logistic regression) and covariates used in the model (HBP . . . hypertension, GE . . . geoethnic structure as determined by principal components 1 and two of PCA analysis, AGE and SEX). PHENOTYPE denotes the case and control setup as described above. CHR identifies chromosomal allocation of the SNPs. P-VALUE shows the p value.

Table 11. The table comprises the official name of SNP according to dbSNP of NCBI (dbSNP RS ID) and the flanking sequence as retrieved from queries to Affymetrix NetAffx™ Analysis Center for the SNPs included in Table 10.

Table 12. The table shows the official symbol (GENE_SYMBOL) and name of the genes (Gene Name) according to Human Genome Organisation Gene Nomenclature Committee for the genes relevant to the whole genome association results of 1908 individuals in Table 10. The relevance here means that one on more of the SNPs listed in Table 10 are located either inside the gene (exon, intron, 5′UTR, 3′UTR) or very close to the gene—as defined by NCBI.

Table 13: List of the SNPs used for geoethnic clustering (14,961 SNP example).

Table 14: List of the SNPs used for geoethnic clustering. (2 000 PCA set).

Table 15: Fraction of the 1904 individuals correctly classified clusters compared to baseline reference of 147691 SNPs.

Table 16: List of SNPs selected using SVM method and their flanking sequence.

Table 17: Mean area under the ROC curve (AUC) and 95% CI for the classification of major cardiovascular complications based on 1000 bootstrap iterations for two models: Only SNPs and SNPs+biomarkers.

Table 18: Mean area under the ROC curve (AUC) and 95% CI for the classification of major cardiovascular complications based on 1000 bootstrap iterations for two models: each biomarkers independently and biomarkers added one by one.

Table 19: List of SNPs selected using bootstrap method and their flanking sequence.

Table 20: List of SNPs comprised in the genes of Table 21.

Table 21: List of genes previously reported to be related to complications of T2D.

Without further elaboration, it is believed that one skilled in the art can, using the preceding description, utilize the following invention to its fullest extent. The following specific preferred embodiments are, therefore, to be construed as merely illustrative, and not limitative of the remainder of the disclosure in any way whatsoever.

In the forgoing and in the following examples, all temperatures are set forth uncorrected in degrees Celsius and, all parts and percentages are by volume, unless otherwise indicated.

Experimental Method

Over the last year several studies have reported significant associations of single nucleotide polymorphisms (SNP) using Whole Genome Scans (WGS) in several areas, including myocardial infarction and T2D, while hypertension appeared to resist genomic resolution at SNP level. Here we analyzed the micro- and macro-vascular complications in type 2 diabetics at the entry of ADVANCE study. The current analysis is restricted to Caucasians to decrease the heterogeneity. Total 1908 subjects were included in this stage of genomic sub-study of ADVANCE. WGS was accomplished using Affymetrix chips (the GeneChip® Human Mapping 500K Array Set, Affymetrix Genome-Wide Human SNP Array 5.0 and Affymetrix Genome-Wide Human SNP Array 6.0) as per their progressive availability with excellent intra complementarity.

This section describes the complete experimental method used by the inventors aiming to discover genetic determinants of complications associated with T2D based on the following main points:

1. Demographic data
2. Genetic data
3. Subject quality filters
4. Genetic data quality filters

### 5. Analyses

6. Phenotype definitions
7. Association designs

### 1. Demographic Data

1.1. Provenance—the demographic data is received from George Institute, Australia as Excel files via e-mail.
1.2. Validation—the demographic data files usually correspond to blood sample shipments and they are checked against those to see if they match. Any discrepancies are recorded and reported back via e-mail.
1.3. Compatibility—the data is further inspected for compatibility with the developed database insertion tool, as some small changes may be required:
   dates format to: yyyy-mm-dd
   patient id column name to: new_id
   single quotes replaced by double quotes
   etc
All these modifications are logged then the files are saved as tab delimited text files for database import.
1.4. Database insertion—the insertion is done using the PrognomixCmd (an in house developed software for interfacing with PostgreSQL and R statistical package) and phenotype insertion command files. The files, their output and the generated insertion SQL commands are logged for traceability. The data is inserted into Prognomix database into person, visit, measure, medication and medical tables.
1.5. Insertion validation—once the data is inserted, the database is queried for several random phenotypes and the output is compared, manually, with the received Excel files.

### 2. Genetic Data

2.1. Provenance—the genetic data is produced by the lab team using the blood samples from Australia and Affymetrix genotyping platform. Several chips are used: the GeneChip® Human Mapping 500K Array Set, Affymetrix Genome-Wide Human SNP Array 5.0 and Affymetrix Genome-Wide Human SNP Array 6.0 and the output consists of multiple data files per sample (subject):
   DAT file—contains the raw scanned image
   CEL file—the normalized image—used to calls the SNP
   XML file—contains experiment related information
   JPEG file—the normalized image compressed as a jpeg file.
All these files are compressed and archived on DVDs.
2.2. Sample quality check—the lab team employs a simple and fast sample quality check (DM).
2.3. SNP call—the CEL files (one per subject) are used to call the SNP genotypes for each subject for all SNPs. We use the software provided by Affymetrix, apt-probset-genotype software (version 1.4 for 500 k chip and v1.8.5 64 b for 5.0 and 6.0 chips), with default parameters corresponding to the chip type. The analysis script and application output are logged for further reference. The output consists in a huge matrix-like text file where the columns represent subjects and the lines markers (SNP) and a summary report file detailing some sample statistics as the genetically determined sex, sample call rate and global call rate. These metrics allows us to estimate the overall quality of the genotyping process. All subjects are called at once, as suggested by Affymetrix (personal communication, Nov. 16, 2007).
2.4. Database insertion—the genetic data database insertion is a two step process: (1) the matrix-like data file is converted in one per subject data file and (2) the individual data files are imported into the database. This process is again done using a SNP insertion script and the PrognomixCmd tool. The data is inserted into snp_genotype table.
2.5. Insertion validation—once the data is inserted, the database is queried for several random subjects and SNP then the output compared, manually, with the matrix-like SNP call file.

### 3. Subject Quality Filters

Subjects may be excluded for subsequent analyses. When the sex determined through genotyping does not match the sex provided in demographic data, a request is made to the George Institute to check the sex. If the re-checked sex matches the genotypic sex, the sex is corrected in the database and the subject is kept. However, if the re-checked sex does not match the genotypic sex, the subject is excluded from further analyses (table issues). Subjects whose genome proportion from the Caucasian ancestral population is below 80% according to the average of 5 STRUCTURE [J. P. Huelsenbeck, P. Andolfatto, Inference of Population Structure Under a Dirichlet Process Model, 2007 175:1787-1802] runs (see below) are excluded from further analyses.

### 4. Genetic Data Quality Filters

SNPs considered for further analyses must pass all the following criteria:
4.1. Call rate—the SNP call rate threshold over all genotype subjects was set at 97%.
4.2. Minor allele frequency (MAF)—the SNP MAF threshold over all genotyped subjects was set to 1%.
4.3. Hardy-Weinberg equilibrium (MAF)—the HWE is computed for all subjects and for each affection separately for cases and controls. For single SNP tests like association we filter on controls HWE p-value using a threshold of 10⁻⁴ when reporting results. For global analyses involving many SNPs (like principal component), we filter out SNP based on all subjects HWE p-value. The cutoff was set to 10⁻⁴ for PCA implemented in EIGENSTRAT [Alkes L Price, Nick J Patterson, Robert M Plenge, Michael E Weinblatt, Nancy A Shadick, David Reich. Principal components analysis corrects for stratification in genome-wide association studies. Nature Genetics, Vol. 38, No. 8. (23 Jul. 2006), pp. 904-909.] and 10⁻⁴ for PCA implemented in R.
4.4. Batch genotype frequency differences—since we used several different chips for genotyping, we noticed a number of SNPs which had completely different calls depending on the chip. To eliminate this situation we compared the chip genotypes frequency separately for cases and controls using a chi-square test and excluded SNPs showing p lower than 10⁻⁴.
4.5. Less reliable SNPs—60 k SNPs were considered less performing by Affymetrix and were masked on the Affymetrix Genome-Wide Human SNP Array 5.0. Although we can access these 60 k SNPs they are filtered out.
Selected SNPs are then checked for the following:
4.6. SNP call clustering issues—we implemented a feature in PrognomixCmd tool which allows us to generate, store and examine the SNP call clusters for all SNPs and subjects. SNPs showing bad clusters according to the following criteria are filtered out:
   no clearly defined clusters
   only two clusters
   two clusters called very far from the third one
   two distinct clusters called the identical
   the calls assigned by algorithm different from those we'd assign by visual inspection.

### 5. Analyses

5.1. Coarse-grain stratification—using STRUCTURE 2.2. All subjects are analysed together. 5 different sets of SNPs were used in order to assess the variability in the results due to the choice of the SNPs. Each set contains around 400 SNPs, chosen according to the following criteria: (1) on autosomes; (2) found in Perlegen, HapMap and Affymetrix data sets; (3) spaced at least 5 MB apart; (4) showing Fst>0.2 according to Perlegen (genome.perlegen.com/data); (4) same strand is used by Affymetrix and HapMap. The 210 founder HapMap subjects are included in the analysis (60 children are discarded). The following parameters are used: (1) 10,000 burnin iterations; (2) 50,000 real iterations; (3) 3 populations assumed; (4) admixture allowed; (5) no information about origin of subjects provided.
5.2. Fine grain stratification: PCA analysis was performed using either in house developed software or the SMARTPCA module of Eigensoft (ref: Alkes L Price, Nick J Patterson, Robert M Plenge, Michael E Weinblatt, Nancy A Shadick, David Reich. Principal components analysis corrects for stratification in genome-wide association studies. Nature Genetics, Vol. 38, No. 8. (23 Jul. 2006), pp. 904-909). The two first principal components (i.e. those that explained most of the variance) were used when PCA was included as a covariate in the association analyses.
5.3. Selection of covariates: We used Random Forest (Breiman L (2001). Random Forests, Machine Learning, 45: 5-32.) to adjust for unknown stratification or confounding factors in the reported associations by selecting covariates susceptible to inflate p-values. Random Forests is a machine learning approach based on classification and regression tree methods which offers the advantage of being robust against overfitting. Every tree is built using a bootstrap sample of all the subjects and, at each node of the tree, a random subset (parameter mtry) of all predictors (covariates) is chosen to determine the best split rather than the whole set of predictors. For each tree, approx. one third of all subjects are excluded from the bootstrap sample, also referred to as “out-of-bag” or OOB subjects, and these subjects are used to estimate prediction accuracy of the Random Forest. The Random Forest method also produces an importance measure for each predictor, which quantifies the relative contribution of each predictor to the prediction accuracy. For each phenotype, we selected covariates based on importance measures estimated from 2000 trees “grown” in the randomForest R package version 4.5-28 (Liaw A, Wiener M (2002). Classification and Regression by random Forest, R News 2(3): 18-22.). Parameter mtry was set equal to the square root of the number of predictors used to grow the forest (default value). We imputed missing values in the set of predictors using function rfImpute implemented in the package.
5.4. Single SNP association—this kind of test was done using the PrognomixCmd tool or the Prognomix Discovery Support tool and one of the four implemented association types:
   5.4.1. binary association—consists in an Armitage trend test for additive, dominant and recessive models, a chi-square or Fisher test for the allelic model and a 2 by 3 chi-square test for the genotypic model. These tests are implemented in the R library assoc by the function snp.chisq and in the PLINK [Purcell S, Neale B, Todd-Brown K, Thomas L, Ferreira M A R, Bender D, Mailer J, Sklar P, de Bakker P I W, Daly M J & Sham P C. (2007).PLINK: a toolset for whole-genome association and population-based linkage analysis. American Journal of Human Genetics, 81.] tool by the function—assoc-geno.
   5.4.2. continuous association—performs parametric or non parametric tests depending on the normality of the analysed trait (tested with a Shapiro-Wilk test) and the model:
      additive model—linear regression.
      dominant, recessive or allelic models—T-test or Wilcoxon test.
      genotypic model—Anova or Kruskal-Wallis tests.
   These tests are implemented in the R library assoc by the function assoc.cont.
   5.4.3. linear regression association for quantitative traits with covariates—uses a linear regression model to test for association for all five models, possibly using covariates. These tests are implemented in the R library assoc by the function assoc.model by specifying a Gaussian regression model and in the PLINK tool by the function—linear.
   5.4.4. logistic regression association for binary traits with covariates—uses a logistic regression model to test for association for all five models, possibly using covariates. These tests are implemented in the R library assoc by the function assoc.model by specifying a Binomial regression model and in the PLINK tool by the function—logistic.
   All four association types involve several similar steps, which can be all done using PrognomixCmd tool or the Prognomix Discovery Support tool and association command files:
      Trait data extraction—the qualitative or quantitative trait data is extracted form database for all subjects or for a subset of them into text files and transferred on the computation server.
      Genetic data extraction—the genetic data is extracted form database, generally for all markers (sometimes only for a chromosome or a list of selected SNPs).
      Test—the test is performed on the computation server using custom developed R library assoc. or PLINK.
      Database results insertion—the results generated by the tests are transferred back from the computation server, and then inserted into Prognomix database or stored using the data storage functionality of the Prognomix Discovery Support tool.
      Result reporting—the best results are extracted from database using predefined or custom defined quality filters and reported into Excel files. The interesting results are further investigated for non-automated quality filters (clusters) as described above.
   All the analysis scripts and the application output are logged for further reference or audit.
5.5. Haplotype association—this test was only performed in genomic regions of interest using several different methods:
   5.5.1. Find haplotype blocks in HapMap CEU population then use the blocks in our population to test for association using HaploView with the default parameters. HaploView: estimates haplotypes using an estimation-maximization (EM) approach then test association using a chi-square test for each haplotype.
   5.5.2. Find haplotype blocks in HapMap CEU population then use the blocks in our population to test for association using the R library Haplo.Stats. Haplo.Stats uses an EM haplotype inference algorithm which incorporates the haplotype phase uncertainty rather than assigning a most likely haplotype to each sample then test for association using a generalized linear regression model.
   5.5.3. Use the sliding window approach implemented in PLINK with different windows sizes inside which the haplotypes are estimated using an EM approach, then the association is tested with a chi-square test for each haplotype.
   5.5.4. Use the tag SNP identification method implemented in ldSelect, which create haplotypes with tag SNPs and test for association using Haplo.Stats as above. ldSelect identifies tag SNP based on linkage disequilibrium statistic r² and a greedy algorithm by grouping SNPs into bins of associates sites where the pair wise r² is above a threshold. One SNP per bin is the tag SNP.

### 6. Phenotype Definitions

Two sets of phenotype definitions were used.
The first set was based on the initial definitions used at baseline of the ADVANCE study and was as follow:
   6.1. Stroke: cerebrovascular accident before or at the entry to ADVANCE (ADVANCE definition: Damage to blood vessels in the brain where vessels burst and bleed or become clogged with fatty deposits. When blood flow is interrupted, brain cells die or are damaged, resulting in a stroke.)
   6.2. Myocardial infarction: heart attack before or at the entry to ADVANCE (ADVANCE definition: The medical term for heart attack, which occurs when the blood supply to part of the heart muscle itself—the myocardium—is severely reduced or stopped, resulting in the death of a segment of the heart muscle.)
   6.3. Albuminuria: albumin/creatinine ratio is equal or higher than 30 μg/mg at the entry to ADVANCE (ADVANCE definition: The presence of albumin in the urine that is usually a symptom of disease of the kidneys.)
   6.4. Complications: Stroke and/or myocardial infarction and/or albuminuria as defined above.
   6.5. No Complications: None of the above complications.
   The second set of definitions was established after inclusion of follow up data obtained during the conduct of the ADVANCE study and was as follow:
   6.6. Controls are T2D that at the entry to ADVANCE study had no history of or were not diagnosed with: myocardial infarction, hospitalization for unstable angina, stroke, transitory ischaemic attack, atrial fibrillation, heart failure, left ventricular hypertrophy, background retinopathy, blindness, macular oedema, proliferative retinopathy, retinal photocoagulation therapy, peripheral revascularization, amputation, estimated glomerular filtration rate below 60 mL/min/1.73 m², and albumin/creatinine ratio higher than 30 μg/mg.
   6.7. Supercontrols are a subset of controls that did not have any macrovascular (cardiovascular death, myocardial infarction, stroke) or microvascular [new or worsening nephropathy (macroalbuminuria—albumin/creatinine do higher than 300 μg/mg; high serum creatinine—higher than 200 μmol/L; need for renal replacement therapy; death due to renal disease), retinopathy (proliferative retinopathy, macular oedema, blindness, history of retinal coagulation therapy] events during the duration of ADVANCE study.
   6.8. Myocardial infarction: Heart attack and/or Q-waves diagnostics of previous myocardial infarction and/or hospitalization for unstable angina
   6.9. Albumin/creatinine ratio: quantitative trait (units μg/mg)
   6.10. Albuminuria: albumin/creatinine ratio is equal or higher than 30 μg/mg
   6.11. Microalbuminuria: albumin/creatinine ratio is equal or higher than 30 μg/mg and lower than 300 μg/mg
   6.12. Macroalbuminuria: albumin/creatinine ratio is equal or higher than 300 μg/mg
   6.13. Estimated glomerular filtration rate: quantitative trait (units mL/min/1.73 m²)
   6.14. Low estimated glomerular filtration rate: values below 60 mL/min/1.73 m²
   6.15. New worsening nephropathy event: macroalbuminuria (albumin/creatinine ratio higher than 300 μg/mg) and/or high serum creatinine (higher than 200 μmol/L) and/or need for renal replacement therapy and/or death due to renal disease) during the ADVANCE study

### 7. Association Designs

Using the first set of phenotypes we performed analysis based on the designs listed below. The results of these analyses are provided in Tables 1, 2, and 3. Table 1 provides the SNPs IDs, details of the phenotypes and covariates used during analysis and p values and odds ratios obtained for each SNP. Table 2 provides the flanking sequence of the SNPs of Table 1 and Table 3 the genes related to the SNPs of Table 1.
Myocardial infarction vs. no myocardial infarction (see Table 1, phenotype=mi):
   Cases: Type 2 diabetic patients with heart attack before or at the entry to the ADVANCE study
   Controls: Type 2 diabetic patients without heart attack at the entry to the ADVANCE study
   Covariates: no covariates, geoethnicity-PCA1
Myocardial infarction vs. no myocardial infarction—patients from region 1 only (see Table 1, phenotype=mi, region-1)
   Cases: Type 2 diabetic patients from region 1 with heart attack before or at the entry to the ADVANCE study
   Controls: Type 2 diabetic patients from region 1 without heart attack at the entry to the ADVANCE study
   Covariates: no covariates
Myocardial infarction vs. no myocardial infarction—patients from region 3 only (see Table 1: phenotype=mi, region-3)
   Cases: Type 2 diabetic patients from region 3 with heart attack before or at the entry to the ADVANCE study
   Controls: Type 2 diabetic patients from region 3 without heart attack at the entry to the ADVANCE study
   Covariates: no covariates
Albuminuria vs. no albuminuria (see Table 1, phenotype=albu):
   Cases: Type 2 diabetic patients having albuminuria at the entry to the ADVANCE study
   Controls: Type 2 diabetic patients without albuminuria at the entry to the ADVANCE study
   Covariates: no covariates, geoethnicity-PCA1
Albuminuria vs. no albuminuria—patients from region 1 only (see Table 1, phenotype=albu, region-1)
   Cases: Type 2 diabetic patients from region 1 having albuminuria at the entry to the ADVANCE study
   Controls: Type 2 diabetic patients from region 1 without albuminuria at the entry to the ADVANCE study
   Covariates: no covariates
Albuminuria vs. albuminuria—patients from region 3 only (see Table 1, phenotype=albu, region-3)
   Cases: Type 2 diabetic patients from region 3 having albuminuria at the entry to the ADVANCE study
   Controls: Type 2 diabetic patients from region 3 without albuminuria at the entry to the ADVANCE study
   Covariates: no covariates
Complications vs. no complications (see Table 1, phenotype=complications)
   Cases: Type 2 diabetic patients having the above defined complications before or at the entry to the ADVANCE study
   Controls: Type 2 diabetic patients without the above defined complications at the entry to the ADVANCE study
   Covariates: no covariates, geoethnicity-PCA1
Complications vs. no complications—patients from region 1 only (see Table 1, phenotype=complications, region-1)
   Cases: Type 2 diabetic patients from region 1 having the above defined complications before or at the entry to the ADVANCE study
   Controls: Type 2 diabetic patients from region 1 without the above defined complications at the entry to the ADVANCE study
   Covariates: no covariates
Complications vs. no complications—patient from region 3 only (see Table 1, phenotype=complications, region-3)
   Cases: Type 2 diabetic patients from region 3 having the above defined complications before or at the entry to the ADVANCE study
   Controls: Type 2 diabetic patients from region 3 without the above defined complications at the entry to the ADVANCE study
   Covariates: no covariates
Stroke vs. no stroke (see Table 1, phenotype=stroke)
   Cases: Type 2 diabetic patients with stroke before or at the entry to the ADVANCE study
   Controls: Type 2 diabetic patients without stroke at the entry to the ADVANCE study
   Covariates: no covariates, geoethnicity-PCA1
Stroke vs. no stroke—patients from region 1 only (see Table 1, phenotype=stroke, region-1)
   Cases: Type 2 diabetic patients from region 1 with stroke before or at the entry to the ADVANCE study
   Controls: Type 2 diabetic patients from region 1 without stroke at the entry to the ADVANCE study
   Covariates: no covariates
Stroke vs. no stroke—patients region 3 only (see Table 1, phenotype=stroke, region-3)
   Cases: Type 2 diabetic patients from region 3 with stroke before or at the entry to the ADVANCE study
   Controls: Type 2 diabetic patients from region 3 without stroke at the entry to the ADVANCE study
   Covariates: no covariates
      Using the second set of phenotypes we performed analysis based on the designs listed below. For myocardial infarction, the results of these analyses are provided in Tables 4, 5, and 6. Table 4 provides the SNPs IDs, details of the phenotypes and covariates used during analysis and p values and odds ratios obtained for each SNP. Table 5 provides the flanking sequence of the SNPs of Table 4 and Table 6 the genes related to the SNPs of Table 4. For kidney disease (qualitative phenotypes), the results of these analyses are provided in Tables 7, 8, and 9. Table 7 provides the SNPs IDs, details of the phenotypes and covariates used during analysis and p values and odds ratios obtained for each SNP. Table 8 provides the flanking sequence of the SNPs of Table 7 and Table 9 the genes related to the SNPs of Table 7. For kidney disease (quantitative phenotypes), the results of these analyses are provided in Tables 10, 11, and 12. Table 10 provides the SNPs IDs, details of the phenotypes and covariates used during analysis and p values and odds ratios obtained for each SNP. Table 11 provides the flanking sequence of the SNPs of Table 10 and Table 12 the genes related to the SNPs of Table 10.
Myocardial infarction before or at the entry to ADVANCE versus no myocardial infarction before or at the entry to ADVANCE (see Table 4, phenotype=MI vs no MI (at entry))
   Cases: Type 2 diabetic patients having myocardial infarction before or at the entry to ADVANCE study
   Controls: Type 2 diabetic patients free of myocardial infarction at the entry to ADVANCE study
   Covariates: no covariates; age+sex; age+sex+geoethnicity; age+sex+geoethnicity+status of being currently treated for hypertension; age+sex+geoethnicity+albumin/creatinine ratio; age+sex+geoethnicity+estimated glomerular filtration rate
Myocardial infarction before or at the entry to ADVANCE versus clean controls (see Table 4, phenotype=MI vs control (at entry))
   Cases: Type 2 diabetic patients having myocardial infarction before or at the entry to ADVANCE study plus any other condition/disease
   Controls: controls as defined above
   Covariates: no covariates; age+sex; age+sex+geoethnicity; age+sex+geoethnicity+status of being currently treated for hypertension; age+sex+geoethnicity+albumin/creatinine ratio; age+sex+geoethnicity+estimated glomerular filtration rate
Myocardial infarction before and/or during the ADVANCE study versus no myocardial infarction before and during the ADVANCE study (see Table 4, phenotype=MI vs no MI (follow-up))
   Cases: Type 2 diabetic patients having myocardial infarction before and/or during the ADVANCE study
   Controls: Type 2 diabetic patients free of myocardial infarction before and during the ADVANCE study
   Covariates: no covariates; age+sex; age+sex+geoethnicity; age+sex+geoethnicity+status of being currently treated for hypertension; age+sex+geoethnicity+albumin/creatinine ratio; age+sex+geoethnicity+estimated glomerular filtration rate
Myocardial infarction before and/or during the ADVANCE study versus supercontrols (see Table 4, phenotype=MI vs control (follow-up))
   Cases: Type 2 diabetic patients having myocardial infarction before and/or during the ADVANCE study plus any other condition/disease
   Controls: Supercontrols as defined above
   Covariates: no covariates; age+sex; age+sex+geoethnicity; age+sex+geoethnicity+status of being currently treated for hypertension; age+sex+geoethnicity+albumin/creatinine ratio; age+sex+geoethnicity+estimated glomerular filtration rate
Albumin/creatinine ratio as a quantitative trait (see Table 10, phenotype=Albumin/Creatinine ratio at entry)
   Covariates: no covariates; age+sex; age+sex+geoethnicity
Estimated glomerular filtration rate as a quantitative trait (see Table 10, phenotype=Creatinine Clearance at entry)
   Covariates: no covariates; age+sex; age+sex+geoethnicity
Albuminuria at the entry to ADVANCE versus no albuminuria at the entry to ADVANCE (see Table 7, phenotype=albuminuria vs. no albuminuria (at entry))
   Cases: Type 2 diabetic patients having albuminuria at the entry to ADVANCE
   Controls: Type 2 diabetic patients without albuminuria at the entry to ADVANCE
   Covariates: no covariates; age+sex; age+sex+geoethnicity
Albuminuria at the entry to ADVANCE versus clean controls (see Table 7, phenotype=microalbuminuria or macroalbuminuria vs. control (at entry))
   Cases: Type 2 diabetic patients having albuminuria at the entry to ADVANCE
   Controls: controls as defined above
   Covariates: no covariates; age+sex; age+sex+geoethnicity
Microalbuminuria at the entry to ADVANCE versus no albuminuria at the entry to ADVANCE (see Table 7, phenotype=microalbuminuria vs. no albuminuria (at entry))
   Cases: Type 2 diabetic patients having microalbuminuria at the entry to ADVANCE
   Controls: Type 2 diabetic patients without albuminuria at the entry to ADVANCE
   Covariates: no covariates; age+sex; age+sex+geoethnicity

### Macroalbuminuria at the entry to ADVANCE versus no albuminuria at the entry to ADVANCE (see Table 7, phenotype=macroalbuminuria vs. no albuminuria (at entry))

Cases: Type 2 diabetic patients having macroalbuminuria at the entry to ADVANCE
Controls: Type 2 diabetic patients without albuminuria at the entry to ADVANCE
Covariates: no covariates; age+sex; age+sex+geoethnicity
Albuminuria at the entry and/or during the ADVANCE study versus no albuminuria at the entry and during the ADVANCE study (see Table 7, phenotype=albuminuria vs. no albuminuria (follow-up))
   Cases: Type 2 diabetic patients having albuminuria at the entry and/or during the ADVANCE study
   Controls: Type 2 diabetic patients without albuminuria at the entry and during the ADVANCE study
   Covariates: no covariates; age+sex; age+sex+geoethnicity
Albuminuria at the entry and/or during the ADVANCE study versus supercontrols (see Table 7, phenotype=microalbuminuria or macroalbuminuria vs. control (follow-up))
   Cases: Type 2 diabetic patients having albuminuria at the entry and/or during the ADVANCE study
   Controls: supercontrols as defined above
   Covariates: no covariates; age+sex; age+sex+geoethnicity
Low estimated glomerular filtration rate at the entry to ADVANCE study versus no low estimated glomerular filtration rate at the entry to ADVANCE study (see Table 7, phenotype=low creatinine clearance vs. normal creatinine clearance)
   Cases: Type 2 diabetic patients having low estimated glomerular filtration rate at the entry to ADVANCE study
   Controls: Type 2 diabetic patients not having low estimated glomerular filtration rate at the entry to ADVANCE study
   Covariates: no covariates; age+sex; age+sex+geoethnicity
Low estimated glomerular filtration rate at the entry to ADVANCE study versus clean controls (see Table 7, phenotype=low glomerular filtration rate vs. control (at entry))
   Cases: Type 2 diabetic patients having low estimated glomerular filtration rate at the entry to ADVANCE study
   Controls: controls as defined above
   Covariates: no covariates; age+sex; age+sex+geoethnicity
Low estimated glomerular filtration rate at the entry to ADVANCE and/or new worsening nephropathy event during the ADVANCE study versus no low estimated glomerular filtration rate at the entry and no new worsening nephropathy event during ADVANCE study (see Table 7, phenotype=low glomerular filtration rate vs. normal glom. filtration rate (follow-up))
   Cases: Type 2 diabetic patients having low estimated glomerular filtration rate at the entry to ADVANCE study and/or new worsening nephropathy event
   Controls: Type 2 diabetic patients not having low estimated glomerular filtration rate at the entry to ADVANCE study and no new worsening nephropathy event
   Covariates: no covariates; age+sex; age+sex+geoethnicity
Low estimated glomerular filtration rate at the entry to ADVANCE and/or new worsening nephropathy event during the ADVANCE study versus supercontrols (see Table 7, phenotype=low glomerular filtration rate vs. control (follow-up))
   Cases: Type 2 diabetic patients having low estimated glomerular filtration rate at the entry to ADVANCE study and/or new worsening nephropathy event
   Controls: supercontrols as defined above
   Covariates: no covariates; age+sex; age+sex+geoethnicity

### EXAMPLES

The invention will be explained below with reference to the following non-limiting examples.

### Example 1

### Geoethnic Clustering

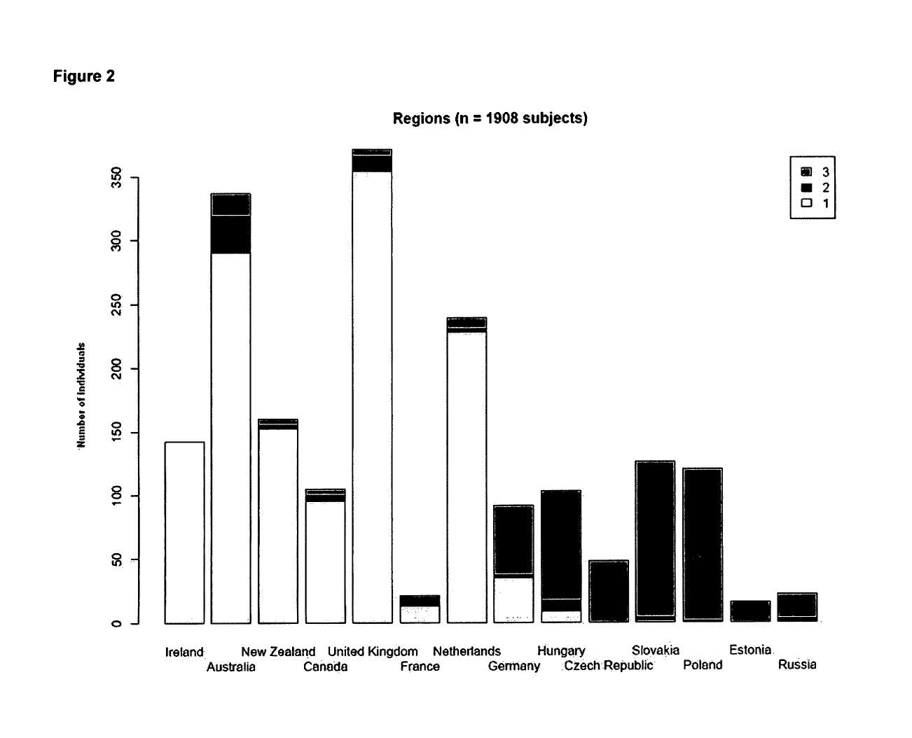

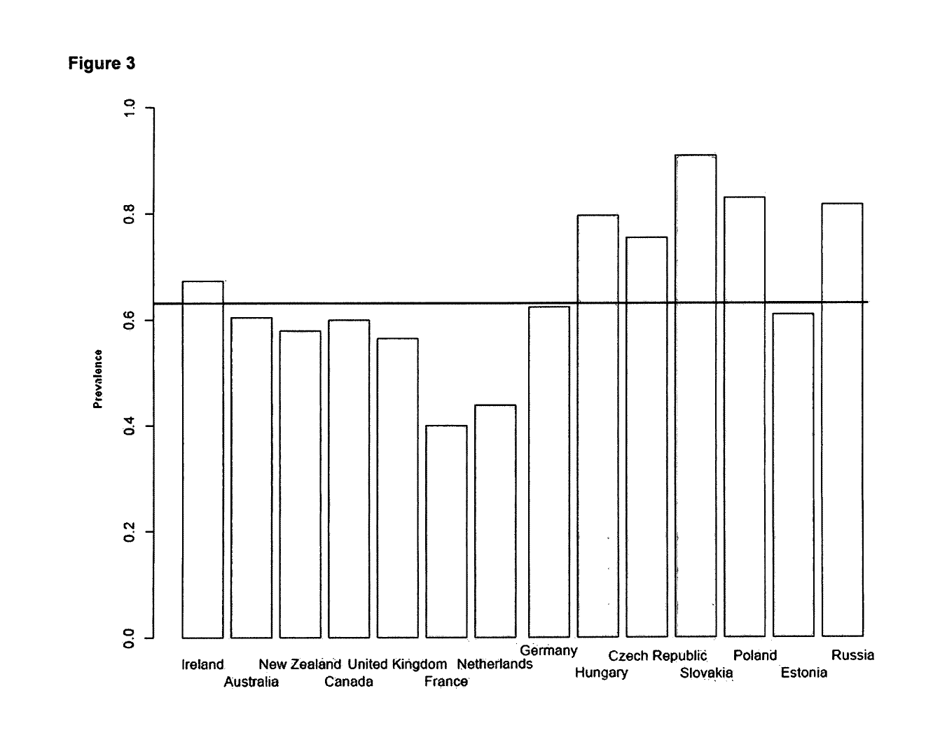

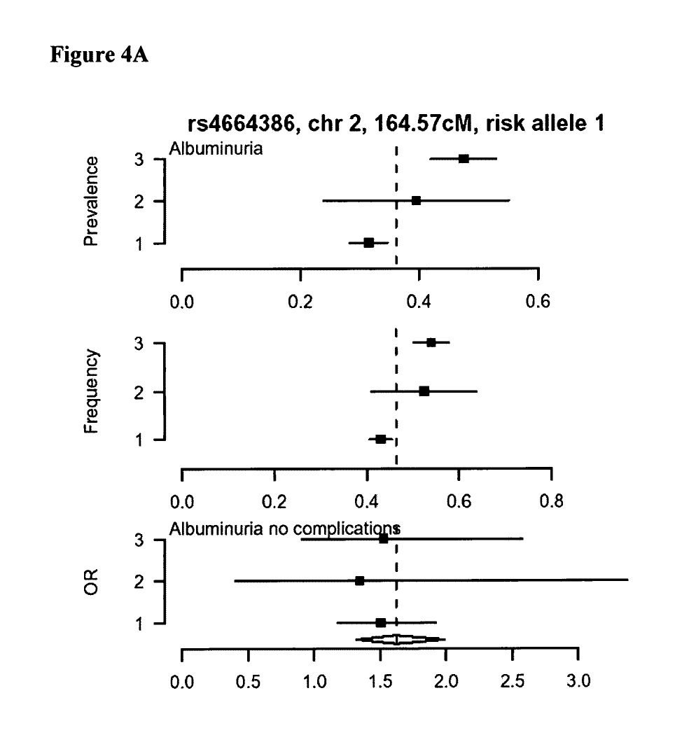

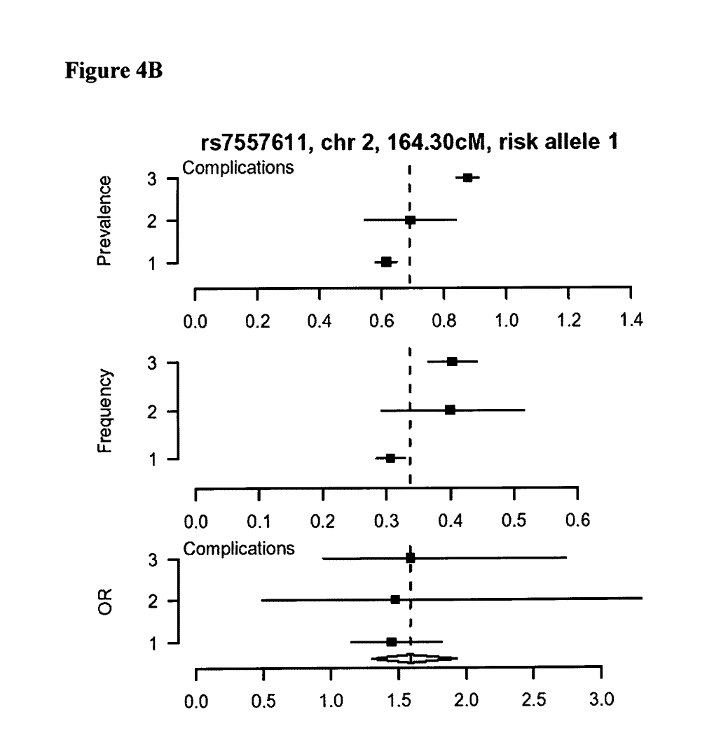

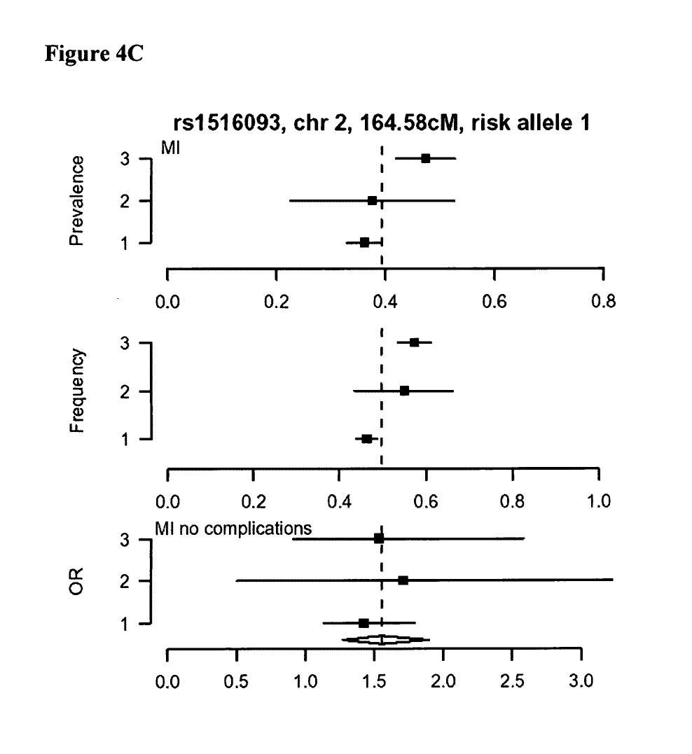

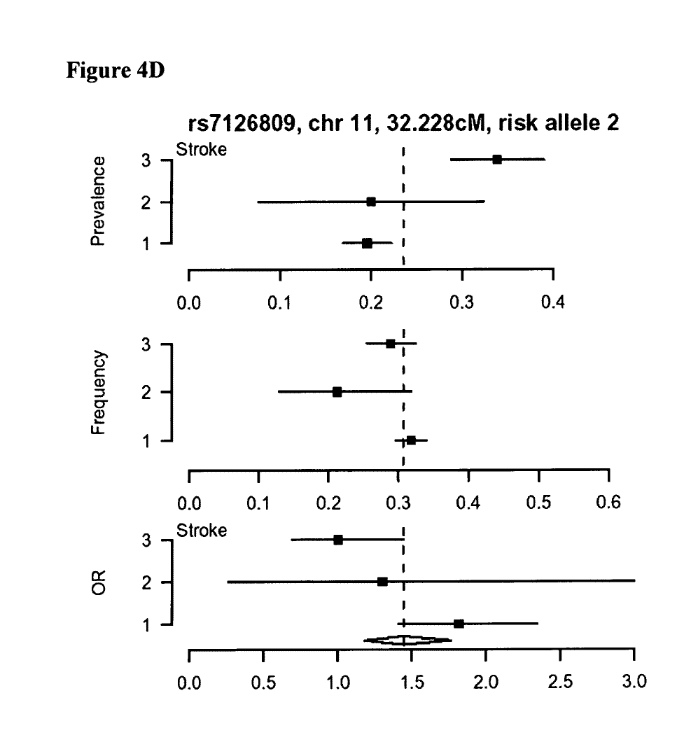

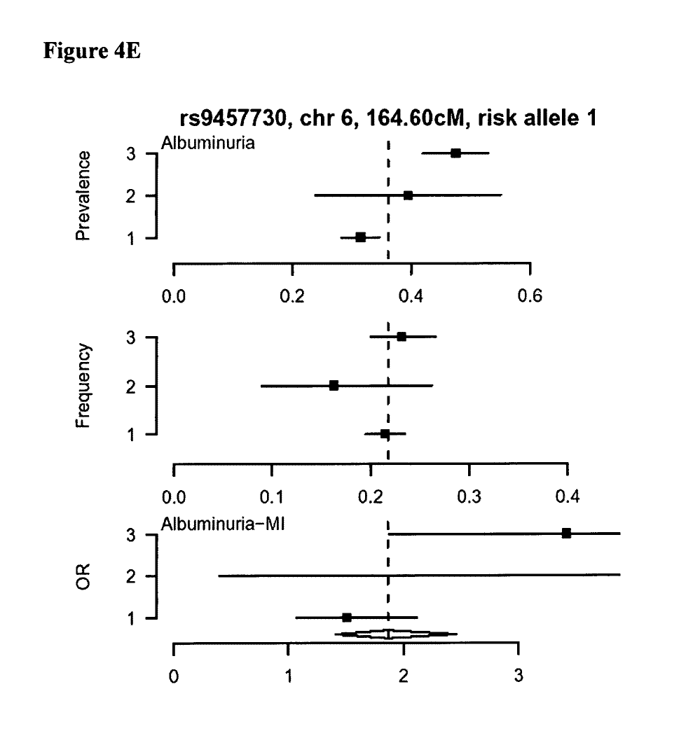

It is has been shown that we can distinguish without any overlap Caucasians, Africans and Asians. Given the density of the currently available genomic markers and the power of our study, we demonstrated that we can distinguish individuals of Caucasian origin within European populations. Our data show that using a subset of 14,961 SNPs (list in provided in Table 13) unrelated to T2D complications, we were able to cluster the individuals into three groups along the PC1 and PC2 axes (FIG. 1): Group 1 (n=1317 individuals), Group 2 (n=80) and Group 3 (n=507). The two predominant populations (Group 1 and Group 3) exhibit a west/east cline in Europe and a majority of the western type (Group 1) were found in Australia, New-Zealand and Canada (FIG. 2). It is relevant to state here that population in Eastern Europe (Group 3) present a higher prevalence of complications such as and not limited to albuminuria, hypertension, stroke and myocardial infarction (FIG. 3). The observed excess of complications in predominantly Eastern regions (Group 3) is mainly related to the higher frequency of risk alleles compared to Western regions (Group 1) (FIG. 4A-C, example of SNPs on chr 2 for albuminuria, all complications and MI). The figure shows same impact in an individual independently of environment, but higher allele frequency in population 3. Occurring less frequently, the increased disease prevalence (albuminuria or stroke) is at least partially dependent on the allele penetrance (albuminuria on chr6 and stroke on chr11) (i.e., the frequency is similar in groups 1 and 3, but the impact on the individual is more severe in his/her genomic in population 1 for stroke and population 3 for albuminuria). See, FIGS. 4 D and E. The test should thus be appropriately tailored to identify the geo-ethnic specificity by including SNPs among the 14,961 identified in our studies. There is a two prone importance for this finding. 1) a public health relevance modifying the public health strategies as a result of the knowledge of the risk in specific population and 2) at the level of the individual and of his or her relatives, where the impact may require specific individualized approach of prevention and treatment.

Further demonstration of the power of the method can be shown by assessing the effect of varying the number of SNPs used on the accuracy of the classification.

The fine grain stratification analysis consisted of the following steps: 430 205 Affymetrix SNPs that are common to Affymetrix Genome-Wide Human SNP Array 5.0 and Affymetrix Genome-Wide Human SNP Array 6.0 were filtered using PLINK SNP filters which consists of a minor allele frequency (MAF) superior to 0.01, a missing individual call rate (MIND) of at most 0.1, a SNP call rate of at least 0.98 and a Hardy-Weinberg equilibrium threshold of 0.001. After the filters were applied, the 350 895 resulting SNPs were scanned for LD using an R-squared threshold of 0.8 and PLINK. The final number of SNPs was 147691 and all of them were fed to SMARTPCA version 7521 of EIGENSOFT 2.0 (Alkes L Price, Nick J Patterson, Robert M Plenge, Michael E Weinblatt, Nancy A Shadick, David Reich. Principal components analysis corrects for stratification in genome-wide association studies. Nature Genetics, Vol. 38, No. 8. (23 Jul. 2006), pp. 904-909) to obtain the principal components number 1 and 2.

The fine grain principal component analysis using only 2 000 SNPs consisted of the following steps: 1) run the analysis for 147 691 SNPs (see above) and then obtain the SNP weights (loadings) for PC1 and PC2 from SMARTPCA; 2) use the PC1 and PC2 weights to derive a combined score as the following: SCORE=absolute value (PC1)+absolute value (PC2) 3) remove the rightmost percentile of the combined score distribution to remove any artifacts that could influence the procedure when using a small number of SNPs; 4) starting from the rightmost distribution of the combined scores, define sets of SNPs as the first 50, 100, 500, 1000, 2000, 5000 and 10000; 5) Run SMARTPCA to obtain the PC1 and PC2 values for every set of SNPs; 6) Run K-means clustering (in house method) on the fine grain stratification of the 147691 SNPs to obtain 3 specific clusters; 7) repeat this procedure for the sets of SNPs defined above (50, 100, 500, 1000, 2000, 5000 and 10000); 8) obtain cluster classification accuracy by comparing the cluster assigned to each individual from every set of SNP versus the reference clusters of the PCA run with 147691 SNPs (see table 15). The lowest number of SNPs which yields 99% classification accuracy is 2000 (listed in table 14).

The models obtained from either 500 to 147691 SNPs could be used to assign newly obtained patients to a geographic location. This could imply geographic-specific conclusions incorporated into our prediction tool.

### Example 2

### Combination of SNPs

Demonstration of the predictive power of the SNP provided in tables 1, 4, 7 and 10 could be made by combining several of those SNP markers selected based on their level of association with diabetes complications and on the frequency of their risk or protective alleles in the population. Several methods are known by those skilled in the art to select appropriate markers.

Particularly preferred are combinations of biomarkers provided in tables 16 and 19.

Prediction models rely on training and testing sets or bootstrap procedures. Two different models of classification were used: logistic regression and support vector machines. Logistic regression is a well known method which models the probability of a binary variable representing the outcome of interest (event vs. non-event) as a function of quantitative and/or categorical predictors. Support vector machine searches for optimal hyperplanes that separate two classes (here cases and controls) by maximizing the margin between the classes' closest points.

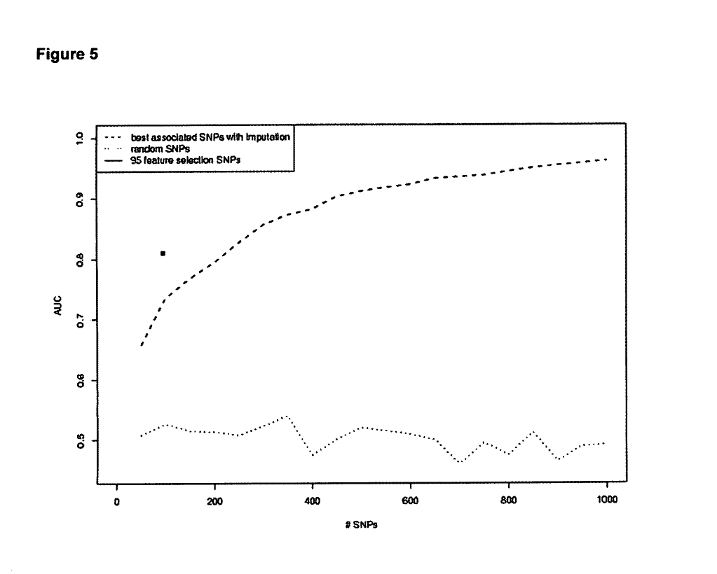

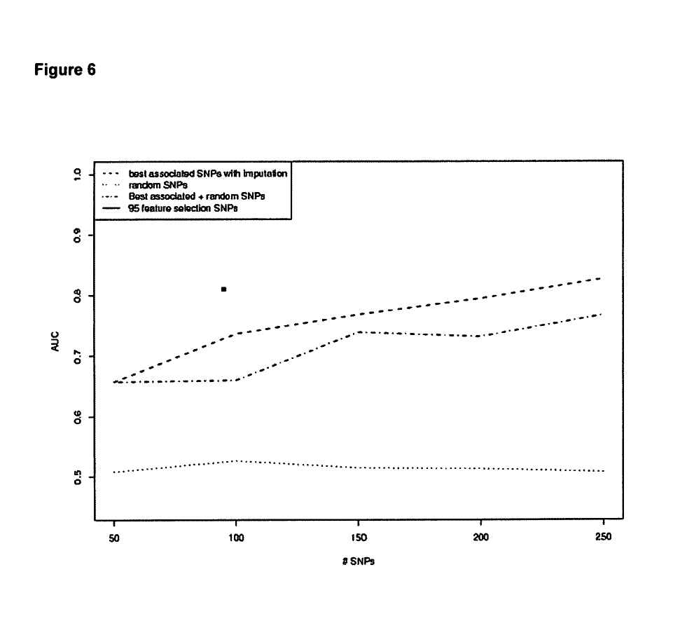

Support vector machines. A subset of discriminative SNPs resulting from GWA association tests should be able to correctly classify the complications related to T2D at an acceptable area under the “Receiver Operating Characteristics” (ROC) curve (AUC) accuracy (0.75) considered between random classification (0.5) and perfect classification (1.0). Based on the hypothesis that a signature found on all observations will classify with acceptable accuracy subsets of individuals composing the whole set, a model was constructed by adding the 1000 most significant SNPs from genome wide associations (p-value) performed on all individuals. Our initial results on T2D complications show that the AUC increases to 0.9 as the number of the best associated SNPs increases to 1000. Association analyses were set to automatically exclude SNPs on the basis of missing genotype rate, with the PLINK—geno option. We included SNPs with at least 98% genotyping rate (2% missing). Given that prediction methods have a hard time or do not deal with missing data, we performed genotype imputation with fastPHASE V1.2.3. [Stephens, M., Smith, N., and Donnelly, P. (2001). A new statistical method for haplotype reconstruction from population data. American Journal of Human Genetics, 68, 978-989] Adding SNPs not-associated to complications never increased the predictive accuracy of the method (FIG. 5). From the top 5 000 associated SNPs in the entire dataset we identified 95 independent SNPs (listed in Table 13) maintaining 0.85 predictive value using INTERACT (Zheng, Zhao. Searching for Interacting Features, In Proc. of International Joint Conference on Artificial Intelligence (IJCAI), January, 2007). (FIG. 6). Using ADVANCE data, we thus demonstrated for the first time significant genomic determinants of vascular complications of diabetes.

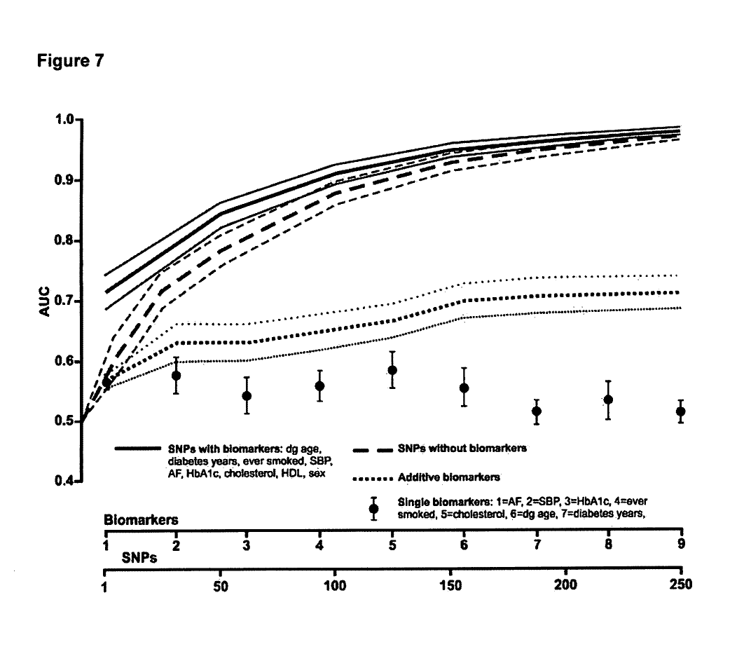

Logistic regression. We used a bootstrap procedure that samples with replacement from the whole dataset during 1000 iterations [1) Angelo Canty and Brian Ripley (2009). boot: Bootstrap R (S-Plus) Functions. R package version 1.2-35. 2) Davison, A. C. & Hinkley, D. V. (1997) Bootstrap Methods and Their Applications. Cambridge University Press, Cambridge]. The AUC reported is the mean from all the bootstrap iterations and a confidence interval was constructed at 95%. The experience was repeated on four different models: only SNPs (top 250 associated SNPs from GWA of myocardial infarction), combined biomarkers and SNPs, only biomarkers added one at a time and each biomarker separately. Examples of biomarkers used for classification include, but are not limited to, age of diagnosis, diabetes duration at baseline, cigarette smoking, systolic blood pressure, atrial fibrillation, glycated hemoglobin (HbA1_{c}), total cholesterol, HDL cholesterol and sex. The FIG. 7 and tables 14 and 15 shows the AUC in function of the number of SNPs and the number of biomarkers by model. The highest classification accuracy of simple biomarkers is achieved by smoking and systolic blood pressure. The predictive accuracy of a model containing all the biomarkers in an additive manner gets an AUC of 0.72. The classification accuracy obtained with 250 best associated SNPs (table 16) is almost similar to the one acquired by a combination of biomarkers and the same number of SNPs (AUC 98%).

### Example 3

### Selection of Patients for Clinical Trials of T2D Drugs

Application of a classification tool in selecting patients with higher risk for T2D complications could dramatically reduce the sample size (and/or the time and cost) required to perform clinical outcome studies in T2D. In our estimations we assumed the following scenario. A randomized clinical trial with two arms is designed to test the impact of novel antidiabetic medication on the rate of cardiovascular events in T2D patients. In one arm, patients receive the usual medication (control arm) whereas, in the other arm (treatment arm), patients receive the novel antidiabetic medication in top of the usual medication. The number of samples required in both arms is such that a difference of 20% between the two arms respective annual event rates will be detected with 80% power at a fixed significance level of 5%. The trial is planned for 5 years.

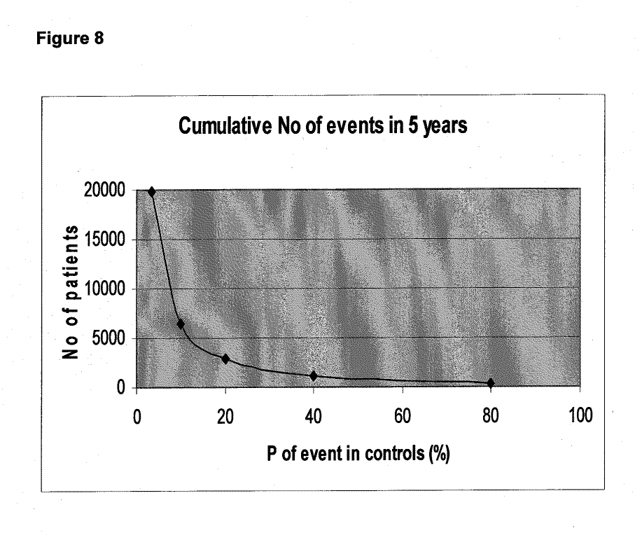

FIG. 8 shows the impact of the annual event rate in the control arm on the number of patients which need to be enrolled at entry. In the graph is shown the observed 3.2% annual event rate of MACE in the ADVANCE trial. Based on a general population of diabetics, the number of subjects needed to achieve the required number of MACE events, without applying any selection criteria at entry, would be ˜60 000 subjects. Using SNPs selected from those listed in tables 1, 4, 7, 10, 13, 14, 16 and 19 of the present invention, with or without other biomarkers, to select patients for such a trial will considerably decrease the number of subjects to recruit in the trial. As an example, increasing AUC from 65% to 85% would reduce the number of required subjects to be enrolled at entry from 11,145 to 2,475 in order to obtain the same number of events during the clinical trial. Similar scenarios may be envisioned for outcome studies for other classes of novel therapies using this approach and, if determining the rate of cardiovascular events is the objective, using SNPs selected from those listed in tables 1, 4, 7, 10, 13, 14, 16 and 19 of the present invention.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions. All publications and patents cited above are incorporated herein by reference.

Lengthy table referenced here
US20110158979A1-20110630-T00001
Please refer to the end of the specification for access instructions.

Lengthy table referenced here
US20110158979A1-20110630-T00002
Please refer to the end of the specification for access instructions.

Lengthy table referenced here
US20110158979A1-20110630-T00003
Please refer to the end of the specification for access instructions.

Lengthy table referenced here
US20110158979A1-20110630-T00004
Please refer to the end of the specification for access instructions.

Lengthy table referenced here
US20110158979A1-20110630-T00005
Please refer to the end of the specification for access instructions.

Lengthy table referenced here
US20110158979A1-20110630-T00006
Please refer to the end of the specification for access instructions.

Lengthy table referenced here
US20110158979A1-20110630-T00007
Please refer to the end of the specification for access instructions.

Lengthy table referenced here
US20110158979A1-20110630-T00008
Please refer to the end of the specification for access instructions.

Lengthy table referenced here
US20110158979A1-20110630-T00009
Please refer to the end of the specification for access instructions.

Lengthy table referenced here
US20110158979A1-20110630-T00010
Please refer to the end of the specification for access instructions.

Lengthy table referenced here
US20110158979A1-20110630-T00011
Please refer to the end of the specification for access instructions.

Lengthy table referenced here
US20110158979A1-20110630-T00012
Please refer to the end of the specification for access instructions.

Lengthy table referenced here
US20110158979A1-20110630-T00013
Please refer to the end of the specification for access instructions.

Lengthy table referenced here
US20110158979A1-20110630-T00014
Please refer to the end of the specification for access instructions.

Lengthy table referenced here
US20110158979A1-20110630-T00015
Please refer to the end of the specification for access instructions.

Lengthy table referenced here
US20110158979A1-20110630-T00016
Please refer to the end of the specification for access instructions.

Lengthy table referenced here
US20110158979A1-20110630-T00017
Please refer to the end of the specification for access instructions.

Lengthy table referenced here
US20110158979A1-20110630-T00018
Please refer to the end of the specification for access instructions.

Lengthy table referenced here
US20110158979A1-20110630-T00019
Please refer to the end of the specification for access instructions.

Lengthy table referenced here
US20110158979A1-20110630-T00020
Please refer to the end of the specification for access instructions.

Lengthy table referenced here
US20110158979A1-20110630-T00021
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20110158979A1). An electronic copy of the table will also be available from the USPTO upon request and payment of the fee set forth in 37 CFR 1.19(b)(3).

1-55. (canceled)

56. A method for predicting a risk of developing a complication which is myocardial infarction, stroke, albuminuria and for declining glomerular filtration in a subject affected by T2D, comprising detecting, in a sample obtained from said subject, at least one genetic feature which is
(a) at least one single nucleotide polymorphism (SNP) listed in Table 1, 4, 7, 10, 16 or 19;
(b) at least one SNP which is in linkage disequilibrium with at least one SNP of (a); or
(c) at least one short tandem repeat (STR) that is in linkage disequilibrium with at least one SNP of (a),
wherein the detection of said genetic feature in said subject correlates with said risk of developing at least one of said complications.

57. The method according to claim 56, comprising detecting a SNP or a STR of at least one gene which is listed in Table 3, 6, 9 or 12.

58. The method according to claim 56, wherein detection of said genetic feature correlates with increased or reduced risk of developing said complication.

59. The method according to claim 56, comprising detecting at least one SNP from the list of SNPs of Table 1, 4, 7 or 10, said SNP being selected on the basis of its p value of association with said complication(s), allele frequency and odds ratio.

60. The method according to claim 56, comprising detecting at least two of said SNPs.

61. The method according to claim 56, comprising detecting at least three of said SNPs.

62. The method according to claim 56, comprising detecting more than three of said SNPs

63. The method according to claim 56, wherein said STR and/or SNP is detected in said patient in a specific geo-ethnic context.

64. The method of claim 63, comprising determining the geoethnic origin of an individual by detecting one or more SNPs listed in Table 13 or 14.

65. The method according to claim 56, wherein the genetic feature is
(a) at least one single nucleotide polymorphism (SNP) listed in Table 16 or 19;
(b) at least one SNP which is in linkage disequilibrium with at least one SNP of (a); or
(c) at least one short tandem repeat (STR) that is in linkage disequilibrium with at least one SNP of (a).

66. The method according to claim 65, comprising detecting at least two SNPs from the SNPs listed in Table 16 or 19.

67. The method according to claim 65, comprising detecting at least three SNPs from the SNPs listed in Table 16 or 19.

68. The method according to claim 56, with the proviso that said at least one SNP is not one of the SNPs listed in table 20.

69. A method for predicting a risk of developing a complication which is myocardial infarction, stroke, or albuminuria and for declining glomerular filtration in a subject affected by T2D, comprising detecting, in a sample obtained from said subject, at least one genetic feature which is
(a) at least one (SNP) from at least one gene listed in Table 3, 6, 9 or 12 or a locus linked to said gene;
(b) at least one STR found to be in linkage disequilibrium with at least one (SNP) from at least one gene listed in Table 3, 6, 9 or 12,
wherein the detection of said SNP or STR in said subject correlates with said risk of developing said complication.

70. The method according to claim 69, with the proviso that said at least one gene is not one of the genes listed in Table 21.

71. A kit for predicting a complication which is myocardial infarction, stroke, albuminuria or declining glomerular filtration in a subject affected by type-2 diabetes (T2D) comprising in one or more packages
(a) an oligonucleotide that specifically hybridizes to a SNP having the RefSNP ID listed in Table 1, 4, 7, 10, 16 or 19; or
(b) an oligonucleotide which is the complement of (a);
and one or more reagents for the detection of said oligonucleotide.

72. The kit according to claim 71, comprising one or more reagents for polymerase chain reaction (PCR).

73. The kit of claim 71, further comprising an RNase.

74. The kit of claim 71, which further comprises one or more reagents for isolation of cells from a sample.

75. The kit of claim 74, wherein said sample is a blood sample.

76. The kit of claim 71, further comprising a DNAse inhibitor.

77. The kit of claim 71, further comprising reagents for sequencing in one or more packages.

78. The kit of claim 71, further comprising one or more nucleic acid microarrays in one or more packages.

79. The kit of claim 71, wherein the oligonucleotide comprises the probe sequences of Tables 2, 5, 8, 9, 11, 16 and 19.

80. The kit of claim 71, wherein the oligonucleotide is
(a) an oligonucleotide comprising at least 80% sequence identity to the oligonucleotide having the RefSNP ID listed in Table 1, 4, 7, 10, 16 or 19; or
(b) an oligonucleotide which is complimentary to the oligonucleotide of (a).

81. The kit of claim 71, wherein the oligonucleotide is
(c) an oligonucleotide comprising at least 90% sequence identity to the oligonucleotide having the RefSNP ID listed in Table 1, 4, 7, 10, 16 or 19; or
(d) an oligonucleotide which is complimentary to the oligonucleotide of (a).

82. The kit of claim 71, wherein the oligonucleotide is
(e) an oligonucleotide comprising at least 95% sequence identity to the oligonucleotide having the RefSNP ID listed in Table 1, 4, 7, 10, 16 or 19; or
(f) an oligonucleotide which is complimentary to the oligonucleotide of (a).

83. The kit of claim 71, wherein the oligonucleotide is
(g) an oligonucleotide comprising at least 100% sequence identity to the oligonucleotide having the RefSNP ID listed in Table 1, 4, 7, 10, 16 or 19; or
(h) an oligonucleotide which is complimentary to the oligonucleotide of (a).

84. The kit of claim 71, which further comprises a control oligonucleotide that hybridizes to the wild-type allele.

85. Use of a kit according to claim 71 for predicting a complication which is myocardial infarction, stroke, albuminuria or declining glomerular filtration in a subject affected by type-2 diabetes (T2D).

86. A method for preventing, treating or reducing the risk of T2D or a T2D related complication which is myocardial infarction, stroke, albuminuria or declining glomerular filtration in a subject in need thereof, comprising administrating to said subject a therapeutic agent which
(a) alters the enzymatic activity or levels of a polypeptide encoded by at least one risk gene listed in Table 3, 6, 9; or
(b) alters the expression of at least one polymorphism disclosed in table 1, 4, 7, 10, 16, or 19; or
(c) alters the specific metabolic or other biologically related pathway implicating the risk gene of (a).

87. The method according to claim 86, wherein the therapeutic agent alters the expression of at least one polymorphism disclosed in table 1, 4, 7, 10, 16, or 19.

88. The method according to claim 86, wherein the therapeutic agent is an antisense oligonucleotide or an siRNA.

89. The method according to claim 86, wherein the therapeutic agent alters the levels or enzymatic activity of a polypeptide encoded by at least one risk gene listed in Table 3, 6, 9.

90. The method according to claim 86, wherein the therapeutic agent is an antibody.

91. A method for identifying a subject for preventive therapeutic action, comprising detecting, in a sample obtained from said subject, at least one genetic feature which is
(a) at least one single nucleotide polymorphism (SNP) listed in Table 1, 4, 7, 10, 16 or 19;
(b) at least one SNP which is in linkage disequilibrium with at least one SNP of (a); or
(c) short tandem repeat (STR) that is in linkage disequilibrium with at least one SNP of (a), wherein the detection of said genetic feature in said subject correlates with the eligibility of said subject for said preventive therapeutic action.

92. A method for characterizing a subject for inclusion or exclusion from a clinical trial, comprising detecting, in a sample obtained from said subject, the presence or absence of at least one genetic feature which is
(a) at least one single nucleotide polymorphism (SNP) listed in Table 1, 4, 7, 10, 16 or 19;
(b) at least one SNP which is in linkage disequilibrium with at least one SNP of (a); or
(c) at least one short tandem repeat (STR) that is in linkage disequilibrium with at least one SNP of (a).

93. The method according to claim 92, comprising detecting a SNP or a STR of at least one gene which is listed in Table 3, 6, 9 or 12.

94. The method according to claim 92, wherein detection of said genetic feature correlates with an increased or reduced risk of developing a complication associated with type 2 diabetes (T2D).

95. The method according to claim 93, wherein detection of said genetic feature correlates with increased risk of developing said complication associated with T2D.

96. The method according to claim 95, wherein said complication associated with T2D is myocardial infarction, stroke or albuminuria and/or declining glomerular filtration or a combination thereof.

97. The method according to claim 96, comprising detecting at least one SNP from the list of SNPs of Table 1, 4, 7 or 10, said SNP being selected on the basis of its p value of association with said complication(s), allele frequency or odds ratio.

98. The method according to claim 92, comprising detecting at least two SNPs.

99. The method according to claim 92, comprising detecting at least three SNPs.

100. The method according to claim 92, comprising detecting more than three SNPs.

101. The method according to claim 92, wherein said STR and/or SNP is detected in said patient in a specific geo-ethnic context.

102. The method according to claim 101, comprising determining the geoethnic origin of an individual by detecting one or more SNPs listed in Table 13 or 14.

103. The method according to claim 92, wherein if said genetic feature is detected in said subject, then the subject is included in said clinical trial.

104. The method according to claim 92, for characterizing a subject for inclusion in a clinical trial comprising detecting the presence of said at least one genetic feature.

105. The method according to claim 92, for characterizing a subject for exclusion from a clinical trial comprising detecting the absence of said at least one genetic feature.

106. The method according to claim 92, wherein the genetic feature is
(a) at least one single nucleotide polymorphism (SNP) listed in Table 16 or 19;
(b) at least one SNP which is in linkage disequilibrium with at least one SNP of (a); or
(c) short tandem repeat (STR) that is in linkage disequilibrium with at least one SNP of (a).

107. The method according to claim 102, comprising detecting at least two SNPs from the SNPs listed in Table 16 or 19.

108. The method according to claim 102, comprising detecting at least three SNPs from the SNPs listed in Table 16 or 19.

109. The method according to claim 92, with the proviso that said at least one SNP is not one of the SNPs listed in table 20.

110. The method according to claim 93, with the proviso that said at least one gene is not one of the genes listed in Table 21.

111. A method for predicting a risk of developing a complication which is myocardial infarction, stroke, albuminuria and/or declining glomerular filtration in a subject affected by type 2 diabetes (T2D), comprising detecting, in a sample obtained from said subject, at least one single nucleotide polymorphism (SNP) having the sequence and/or the RefSNP ID set forth below:

| Serial | | |
|---|---|---|
| No. | Flank | RS ID |
| 1 | GGCCCATTGAGCTCCA[C/T]GCTCCGTTTGTCCACA | rs9680927 |
| 2 | GGGAGAGGAGATCTTC[A/G]CCACTTAAAATAACTA | rs17237063 |
| 3 | GGGTGGTTTCTAGATA[C/G]AGAATGAGATTATTTC | rs290254 |
| 4 | GTGAAGTTGTGTATAA[C/T]AGTCTTCTACAGGCTC | rs6817915 |
| 5 | GTTAAGCTCTGCATAC[A/C]ACATACCATTTGTCAC | rs17764096 |
| 6 | GTTATCTGTCTTTGAA[A/G]AGCATATTACTGTCTT | rs981862 |
| 7 | GTTTTCATCAGTATAA[C/T]GGGAACGATCATGGTA | rs1885290 |
| 8 | TACGATCATCTAGGAA[A/C]CTGTTGAAAATGCAGA | rs7983776 |
| 9 | TACTCTCAAAATGACC[G/T]GGGTGGTGAATCGAAC | rs7838821 |
| 10 | TACTTCTGAAGCATGA[C/T]GCTAGAGTGGAAAGAA | rs16937197 |
| 11 | TCATTTATTAAAACAC[A/G]GCATGATTAGACCACT | rs1161262 |
| 12 | TCCTTATGAGCCTTGC[A/G]GACAAAGAGGTATCCT | rs1438698 |
| 13 | TGAGTTAAGCACCTAA[A/C]AGGAGTTTGCTTCCAT | rs2789416 |
| 14 | TGAGTTGGCTGAGGCC[C/T]ATGTTAGCAGATGGGG | rs4877934 |
| 15 | TGATGCCTGCACAGCA[C/T]GGCGGTTGGAATTCAT | rs7236339 |
| 16 | TGGAACGTAGTTAGGC[A/G]CATTAGTCATGAGATA | rs12641411 |
| 17 | TGTTGATGTGACTCTA[A/G]GATTAGGCAGTTGCTG | rs1337322 |
| 18 | TTAAATAAGCCTTACA[C/T]AAAAAACTATCAACAA | rs2481628 |
| 19 | TTCACCTCTTGACTTG[A/G]AGCCTTATAAACTAGA | rs7908867 |
| 20 | TTGAGCAAATCTTTTA[C/T]GGCAGTTTTCCTCATA | rs17303597 |
| 21 | TTGCCCAGCAGTCAGC[C/G]CTGTTTTTTCTGAAGG | rs284851 |
| 22 | TTGGTGGTACTCTACA[C/T]ACATGGCTGCAAATAG | rs2596929 |
| 23 | TTTAGAAACCATGAGA[A/G]GTGAGCATCAGTGGAG | rs16889712 |
| 24 | TTTCCTAAACTCATCA[C/T]GGCAAGAACATATAAC | rs10801633 |
| 25 | TACAAATACTTTTTAA[C/T]GCCACCTGGCCAATTC | rs2824431 |
| 26 | GGAGTTGGATAGCAAT[A/G]ATATTTAACAGTCATT | rs6782967 |
| 27 | TTGCAGGGTTTCTGTA[C/T]AGTAATGCACATTTCT | rs7632456 |
| 28 | TGGTGCTTTCTGGAGA[A/T]AAAACGCAGCCGTTGC | rs16874145 |
| 29 | AGCTCTTGGGAATCTC[A/G]GAGACAGAGTTATCCA | rs2051549 |
| 30 | CAAGTGACCTAGCTGA[C/G]AAGATCATACATTTGC | rs7751503 |
| 31 | GACATTTCAAACGTTG[C/G]AGTAAAAATTGACATC | rs7921166 |
| 32 | AATGAAGCCAGATTGA[A/G]TAGGTGTTATAATTCC | rs4688486 |
| 33 | AAGAAAAACAGAGGAC[A/G]GGAATTACACAGGTCC | rs6554923 |
| 34 | AGCATTTTTGAGTGCA[C/G]AGGTAGGCAGGAAAGA | rs9984988 |
| 35 | AAAATTTGCTTCAGAA[G/T]AAGAGTAAAACTGTTG | rs16917845 |
| 36 | AAATGGCCCAGGCTGC[A/C]AAAAAGGGGAGCAGCA | rs7195806 |
| 37 | AACATTCTTGTGTAGG[C/T]CCAGAAAACGGATTTA | rs6432655 |
| 38 | AACTGATTTTGAAGAC[A/G]AATACGACTCTAGTGT | rs17821231 |
| 39 | AAGGTGGCAGAACCGC[A/G]GAAATGCAGGTTTCTC | rs7046184 |
| 40 | AAGTAGCTTAGATTGA[A/G]GACCAGTGGAATTGAA | rs16960495 |
| 41 | AAGTGAACAAAAACAT[A/G]AGCCAAAGACAATGAC | rs10081463 |
| 42 | AAGTGAGTCTCACAGG[C/T]ATCGATGGGGCAGCAT | rs17059951 |
| 43 | ACCTGGAAACATATGA[C/G]CTTTTAGTGTATGATG | rs12295814 |
| 44 | AGAAACTTTCAAGTAC[C/T]GTAATAGCTTTCAGTC | rs679992 |
| 45 | AGCACTTTGCTCTTCC[G/T]CACCAATGCAGTACAG | rs2936535 |
| 46 | AGGCCTCACAATTAAA[A/G]CTGACTACAAAAAGAA | rs168965 |
| 47 | AGTAACATCATGAGAC[A/G]GAACTTTTATTTTCTT | rs474068 |
| 48 | AGTTTTAATGTGAACA[A/G]TAAACTGGAGATCTTG | rs2968046 |
| 49 | ATCAAATGTACAAAAA[G/T]GCTTCTCAAGAACATT | rs10023214 |
| 50 | ATCTGACAACACTGCA[A/C]ATTTATGCTGAATACA | rs653709 |
| 51 | ATGTGAAACTGAAGCA[G/T]AGGAAAATACTTACAT | rs6095325 |
| 52 | CAAAGGCTTTGCAAGA[A/G]AAAACATCCACTTTCC | rs273487 |
| 53 | CAAATACAAGTCTACA[C/G]AAAACTGAATAAAATT | rs7662513 |
| 54 | CAAGATAGTAGCATAA[A/G]TAAGCAGCAGGTCTGT | rs1167667 |
| 55 | CACTTAAGTTCAAAAC[A/G]CAATATCATAAACCAA | rs4415071 |
| 56 | CAGCACCAGCTCTGGG[A/G]AGGAAGAGATAAGCTG | rs7067780 |
| 57 | CCCAAGAAGTTCTCAA[A/G]GTTGCAGGCTCAATAC | rs3777478 |
| 58 | CCCCAGTGAGGAGGGA[C/T]GGTCATTTCTGTGATA | rs4774501 |
| 59 | CCTGACTTGACTTAAA[C/G]GTATAAAAGAGGAAGT | rs10168777 |
| 60 | CCTTCTTAATCTCATA[C/T]GGTTAGCTTTTGTTGG | rs9432839 |
| 61 | CTCCTCCTTGAGCACA[C/G]AGTTGAAGCTTGCAGG | rs4531505 |
| 62 | CTCCTCTTTTTAACCT[A/G]AACTTCATTGCCTGAT | rs4810081 |
| 63 | CTTGCTTTTAACCCAA[C/T]TTCTTAATTCAGAACA | rs17032728 |
| 64 | GAACACAAATCAAGAA[C/T]GAGTGCAATTTTCTGG | rs1157157 |
| 65 | GAGACCATAAAGTCTA[C/T]AGGTTGGAGTGTTGTT | rs6592344 |
| 66 | GATGGGAAGTGTCAGA[C/T]ATCAGTTCCACAACTC | rs10911725 |
| 67 | GCACCCTCTGAAATAA[C/T]TCTGCAAACCACTGGG | rs16948985 |
| 68 | GCCAGGAAGAGTTAGC[A/G]AGTAGAATACTATAAT | rs2502845 |
| 69 | GCCTTTCTTGGCAAAA[C/T]GAATAGAAAGCCATCT | rs1361473 |
| 70 | GCGTCGTAACAACTGC[A/G]ATGTCTTATCTTCAGT | rs11805709 |
| 71 | GGAAGGAATGCAAACA[A/G]GAGAAGGGTGTTCAAC | rs1535900 |
| 72 | GAAAGCTTTATCTACC[G/T]GGAACATTAGCATTTC | rs1035704 |
| 73 | TGAATCCTGGTCCTTC[A/C]ATCTGGCGTCATTTTA | rs1450827 |
| 74 | TTGCAGTGACTATCAA[C/T]GTGAGTCCAAATTCCC | rs1464435 |
| 75 | ACCTTCCCTGTATGAA[C/T]GACTCCTCAGAGTAAC | rs2837017 |
| 76 | AATAACTCTCAAAAAA[C/T]ATGGTTTCTCTCTTTC | rs10509870 |
| 77 | AATGGCCTTTATTACA[C/T]AGAGAAGCTTTCCTTC | rs619192 |
| 78 | CAAATCAGGGCAAAAA[G/T]TCAACAGCTTCTGCCC | rs1389798 |
| 79 | CCTCGGGCAAGCCCCA[C/T]AAGCACTGTGAATATG | rs16871059 |
| 80 | GGTGAATGAGTTCTGA[C/T]CTTTCTTCTATAATTT | rs2782343 |
| 81 | TAGGTCTATGAAGTTA[G/T]GTTTTCAGGAGGGCTA | rs9345112 |
| 82 | TATATTTAATAGTTAA[C/T]TCCATTTTTGCAGCTT | rs3766623 |
| 83 | CTGTCAAATCAACTCA[C/T]CTTCAACAAAGGAGTC | rs7501838 |
| 84 | TCTGTGAGAATAGAAA[A/G]TGAATCTTTTGGGAAG | rs9296043 |
| 85 | GTACCATGACTATAGA[C/T]TCCTCAACCATCTCTT | rs1612517 |
| 86 | TATTAACTTAAACACC[A/G]ATTTGCTCAAATTTCT | rs2829290 |
| 87 | AACATCCGGTATGGTA[C/T]GACACAGCGGGTTTGG | rs667897 |
| 88 | AGAGAATTGTTTATGA[C/T]CAGGAATTTGAGGCCA | rs4266861 |
| 89 | AGTCTGTCCCTAAAGC[A/G]CAGAGTTGAACAAGGA | rs1674241 |
| 90 | ATACTTAATTTTTCCA[A/G]TATGGTCTAGGTTAGA | rs9502593 |
| 91 | CAAAATCATGGCTCCA[C/T]CTCTGTGATATTTTGA | rs2683690 |
| 92 | CACATGTGGGTCGTCA[A/G]CTGCCATCAGGAGTAG | rs12973797 |
| 93 | CATGTCCAAATGACTA[C/T]CTACAGTTCAAACTGC | rs11183154 |
| 94 | CTTGCCTAAGCCTCTG[C/T]ATCTGTCCTCAGCACT | rs3744226 |
| 95 | CTTTACGGTAGCGAAA[A/G]TAGCTGACGCACATAT | rs6926642 |
| 96 | TCTCACTCTCTGACTC[A/C]CCATTTTAATGAGCAA | rs13353636 |
| 97 | CATAATACAGAATAGA[A/G]CTGGAATCCAAGTCGA | rs11175003 |
| 98 | ACCAGTTTAGCTAGAA[A/C]TATGATATAAAAGAGG | rs4708456 |
| 99 | GGATTAAGAATATACA[C/T]GTAACAGCTAAAACTA | rs10745938 |
| 100 | GGCCCAGGAGACCCTA[A/G]ATCTTTGTATCTTTTC | rs11211590 |
| 101 | GGCTGTTAGTACCCAA[G/T]GGCAACAACATGAATG | rs6508322 |
| 102 | GGGCAATGTTTCCATC[A/C]ACAAGAGATGGGCGCA | rs4610770 |
| 103 | GGGTAAAGATAGGTCA[C/G]ATGAAGTAATCTCCGC | rs1507216 |
| 104 | GTAAACTTGTCTCTTC[A/G]CTGTAGCTGCTCTTAG | rs11630720 |
| 105 | GTAATGTGTCTGTTTA[C/G]ACCTTCTGCCCATTTT | rs3909441 |
| 106 | GTCAGGGGTTCTCAAA[C/T]GGATTTCAGGACCCTT | rs2602707 |
| 107 | GTCATTGCTCAGTAAA[C/T]TCTAGATCCCATCCAA | rs1502060 |
| 108 | GTCTCAGTCTCTAGAC[C/G]AGGTATTCTCAGATTC | rs12443955 |
| 109 | GTGATTTCTTCTTCAC[C/G]ATCTGAGCGAAGGCAG | rs4330350 |
| 110 | GTGGTTGATACACAGA[A/T]GTTCACTGTATTGTAG | rs4574238 |
| 111 | GTTAAATAGGGATTTC[A/G]ATTGCCAAAAAAACCT | rs9969364 |
| 112 | GTTGCCTGTAAGCCTA[A/G]GTTTGCTTGTTCTTTC | rs10779158 |
| 113 | GTTGGTGACAGCTTAA[C/T]TGATGATGCATAGTGG | rs17109313 |
| 114 | GTTTTCATCAGTATAA[C/T]GGGAACGATCATGGTA | rs1885290 |
| 115 | TAACAGCTTAGAGGAC[A/C]GATGATGGTGTCAGAT | rs4761350 |
| 116 | TAACATTTGGTATTAA[A/G]ATATGTTCTGGCATGG | rs3096130 |
| 117 | TAAGCACAATATTGAA[C/G]TATAAAAGACATCTTG | rs13316352 |
| 118 | TACAAATAGGACATCA[C/T]GAAAACTGCATCAAAT | rs7966105 |
| 119 | TACTCTCAAAATGACC[G/T]GGGTGGTGAATCGAAC | rs7838821 |
| 120 | TAGAAATATCTCAAAA[A/T]TGGAGAGCAAAGGAGA | rs1319458 |
| 121 | TAGATGGTATTGATAC[A/C]GAGGTTAAAGAATCAG | rs9973427 |
| 122 | TAGCCCAACGGATATC[A/C]ATGAAATACTTTGCTT | rs16975959 |
| 123 | TATAGTTAAAGGTTAA[C/T]GGCAAATACTCATCTC | rs6444003 |
| 124 | TATATTACAGCATCTA[C/T]GAGTCGATAAATTTAT | rs6572094 |
| 125 | TATCATCAGCTACACC[A/G]AACACTCAAGTGTAGC | rs12814669 |
| 126 | TATGTAATCTTACAAC[A/G]CGAGTAGTTCTCTGAA | rs9850420 |
| 127 | TATGTATAACCTTGAA[C/G]TGTGAAGGCCAGGATC | rs11054878 |
| 128 | TATGTTCTCTACAGTC[A/G]AATTAAATGAAGAAGA | rsl1189836 |
| 129 | TCATGTGTTCCTATAC[A/G]AGGCCTTTTACAGACT | rs4942093 |
| 130 | TCCCGAGCATCAATAA[A/C]GTGTGTCATGATGTAA | rs11220561 |
| 131 | TCCTCCTTCTGTTTAA[C/T]ACATTTTTCCATCTCA | rs17770541 |
| 132 | ATGTTGACAATTTCCC[A/G]CTATTTTGAAGGACAT | rs1540373 |
| 133 | GAAAAAAATACTGAAG[A/G]AGAGTAGTTCAGGGTG | rs783250 |
| 134 | TCTATTTCTTGTTGCC[A/G]CCAATACCATGATCGA | rs9317050 |
| 135 | TCTCACTCTCTGACTC[A/C]CCATTTTAATGAGCAA | rs13353636 |
| 136 | TCTCTGTGGCATCTGC[A/G]CGGTAATAAAATTTAA | rs11050429 |
| 137 | TCTGATTGACTAAGCA[A/C]AGATAAACCCTGGAGT | rs1864329 |
| 138 | TCTTAATGCACTCTCA[A/T]AAATAAACAAAAGGAG | rs10940152 |
| 139 | TCTTTACTGAGGTCAC[A/G]ACATCTCAATTTTGTG | rs12432351 |
| 140 | TCTTTGACACAGGCTT[A/T]AAAAAAATAAAGAAAG | rs10746342 |
| 141 | TGAATGAAGGAGATGG[A/G]ACTCAGGCCTGCTGTG | rs8078319 |
| 142 | TGACTCTAGAATCAAG[A/G]ACTTCTATATATGCTG | rs1229979 |
| 143 | TGAGGTTGACAGGAAA[C/T]GGCTGTGCATAGATCT | rs12227177 |
| 144 | TGCATCCAAGGGCTGA[C/T]GAATATTTGCAACACC | rs4952521 |
| 145 | TGGTGTTTTCTCATCA[A/C]AGATCTGTTCAAATTA | rs1558531 |
| 146 | TGTATAAATTCCTAAC[G/T]ATACTGCAGACACCTC | rs7957382 |
| 147 | TGTCTAAGTGTGTGCA[A/T]ACCCCAACAGAAGGAA | rs17316202 |
| 148 | TGTCTCTTCCGGTAGA[C/T]AGCAGTAAAGCCTGAT | rs693253 |
| 149 | TGTGAAAGTGCACACA[A/T]CTGTGAGTGATTAAGC | rs1014917 |
| 150 | TGTGATCCATTGTATC[C/T]AGCTCACCTGTTGTTT | rs2568491 |
| 151 | TGTGGGCAAGTCACAC[A/T]CATGCAAATATGTGAG | rs4920424 |
| 152 | TGTTAAATAAATAAAC[C/T]AGGCAGAGAAAGACAA | rs1154436 |
| 153 | TGTTGTAATGTTAGAA[A/G]AGTAGAACAGCTTGTG | rs7795090 |
| 154 | TGTTTTACAAATAGTA[A/C]CCTTTACAAATAGTAA | rs9592445 |
| 155 | TGTTTTCATTATGCTA[A/T]GTACAAGATTGTGCTA | rs2960744 |
| 156 | TTAACATCGAAACATG[G/T]AAGTCTCAGTTACACA | rs11197169 |
| 157 | TTACTACTAAATAAAA[C/T]GTCGTAATGAAATTCA | rs17750253 |
| 158 | TTAGAAACCAGCCACA[A/G]CATTTGGGCACTCCAC | rs1558533 |
| 159 | TTAGAACCTCACAGAA[A/G]TAATTGCTGCAATCAA | rs8035561 |
| 160 | TTATCTACTGATTTAC[A/C]CAATTTACAGAGCCAC | rs9363771 |
| 161 | TTATGTGGATTAAGAT[A/G]ATAAAGTGATCTTTAT | rs7616559 |
| 162 | TTATTAAACATGATAC[A/C]CAAAGTACAGTGAAAA | rs7843358 |
| 163 | TTCATCTGAATCTCCT[C/T]AGATGTCCCTCCGTGT | rs470588 |
| 164 | TTCATTTTCACTGACC[C/T]AAGGAGGAATAAGACA | rs6763712 |
| 165 | TTCTACACGATGCAAT[G/T]AAGGCTTACTGCCATT | rs17151705 |
| 166 | TTCTCCACTGGTAAAC[A/T]CTTAGTCTCTGTCTCA | rs2935260 |
| 167 | TTGAAGACAGTGCTCA[A/G]AGTAAGTGCAAAGAGG | rs10748425 |
| 168 | TTTAAAAAACATCCCA[C/T]TGACTACAAATGGATG | rs33612 |
| 169 | TTTCAGACATGGTTGT[A/C]AAGAGTCAGCTCCTTG | rs203948 |
| 170 | TTTGTTTCTCTTTCAT[A/G]ATAAGGACTGGATCTT | rs4947845 |
| 171 | TTTTCTTGGAAGTATC[A/G]ATAAATGTGTATGTAA | rs4691961 |
| 172 | ATCTCTAACTGGTGAT[A/G]AAGCATCATGAAATTC | rs2205895 |
| 173 | TCACACTGGGATGGAA[C/T]TATGGAACCTATGCCC | rs12504640 |
| 174 | TGAAGAGGAGAAAAAC[A/G]CTACATAACGTGCTAA | rs4600972 |
| 175 | TTAATGGTTTTGGCAA[C/T]ATGCAATACTCATGTC | rs1455024 |
| 176 | AAGGTCACCAAGTTGA[A/G]CATGTATATGGCACAG | rs1413896 |
| 177 | TCAGTCTCTAAAGTAA[C/T]CAAGTCAATTCACTAT | rs3779258 |
| 178 | AGAAAATCGCGTAAGA[A/T]GTCAATATGCTGTTTA | rs4737321 |
| 179 | ATGGTGCAGACATAGA[A/G]CATTATTAGCCATTAT | rs7957503 |
| 180 | TGACTTAGGAGTCAAA[A/G]AGCATGGATCCTACTG | rs7493192 |
| 181 | ATTTTAAACGGTAAGA[G/T]AATTTTCATACACACA | rs10510115 |
| 182 | TCCCTCCTTCTTCCAG[A/G]CTCTAACCGCCAGCTA | rs11160 |
| 183 | AAGAAAAACAGAGGAC[A/G]GGAATTACACAGGTCC | rs6554923 |
| 184 | TAAACACTCTCATGGA[A/G]CATCAGTGTCAGACCA | rs1876681 |
| 185 | ATAAATAAAATGCCAT[C/G]AGTATTACCTACTGCT | rs1482220 |
| 186 | GGTACTACTTGACTTT[C/T]AACATTATACACATGA | rs511298 |
| 187 | AAAAATAATAAGCCTA[C/T]CTTATCTCTCTACATG | rs4937159 |
| 188 | AAACTAAAGCCATTAA[A/G]GTGCGTATAAAATTGA | rs9346035 |
| 189 | AAACTGTGGCTCCTTT[A/G]AAAGAATAGTTTCTGA | rs10989294 |
| 190 | AAAGGGCATGAACTCG[A/T]CCTTTTCCATGGCTGC | rs10865809 |
| 191 | AAATACTATACACTGG[A/C]ACATAAAGAACATATG | rs7982376 |
| 192 | AAGAGACAAAGAGAGA[C/T]AAAGTAGTCAAATGAC | rs12622278 |
| 193 | AAGATCACATGTGTTA[C/T]ACCTTAACGAAAAACT | rs10880791 |
| 194 | AAGCCCAGCAGCTTGA[A/G]ACATAAAGCGAGGTGT | rsl65670 |
| 195 | AAGCTAAGCATTTTGA[A/G]AACAGCTTCTGCCTTT | rs11636758 |
| 196 | AAGCTCACAGAATTTA[A/T]CACACAACTCAGGGAC | rs1566659 |
| 197 | AAGGGCAACTGGAAGA[G/T]GAAGAAGAAGGAGAAG | rs914993 |
| 198 | AAGTGAGGCTACAAAG[A/T]CATGGGAGTTAAATGA | rs17701966 |
| 199 | AATAAACAAATTAATA[C/T]CTTGAGCCTGTTACAG | rs1924664 |
| 200 | AATCTAACACAGCTCC[A/G]AGAATTTTCTTCTGGT | rs12750223 |
| 201 | AATTCAAATGATGCAC[A/C]CATAATATGTTCTCTA | rs9524716 |
| 202 | AATTTTGAGGTGATAC[A/T]AGCATTTAGTCCAAAG | rs12458118 |
| 203 | CATAATACAGAATAGA[A/G]CTGGAATCCAAGTCGA | rs11175003 |
| 204 | ACAAGTGTAACAGAAA[A/T]GGAATGACAGAAAAAA | rs4235846 |
| 205 | ACACAGTTGCTGAGTC[G/T]GAAGAGGGGGAAAGCT | rs1558532 |
| 206 | ACAGCTTCCATTTTAC[C/T]CATCATCCCCATTTTG | rs17334370 |
| 207 | ACAGGATCTAGTGCTC[A/G]ATTGCAAGCAGGTGAC | rs6955265 |
| 208 | ACATTTTTAAAGTAGA[A/G]TAACGCAATATTAATA | rs10159959 |
| 209 | ACCAGTTTAGCTAGAA[A/C]TATGATATAAAAGAGG | rs4708456 |
| 210 | ACCCCACTGAAAAATG[G/T]AAGTCAACACCGATCC | rs2145699 |
| 211 | ACCCGGGAAGTGAAGA[A/C]AGTGCAGTGAACTGTG | rs2372412 |
| 212 | AGCTCTGCAGACCTGA[C/T]AGATGTGGCAAATACA | rs7302315 |
| 213 | ACACGTAATTTACTTC[C/T]AACTGTTATCCAAGGA | rs1545741 |
| 214 | ACGTGGTATTTCTTTC[A/G]AAGCCGGTTGCAAACC | rs16980283 |
| 215 | ACTGCAAATACATTTA[A/C]AGGAAGGAAGAATATT | rs73056 |
| 216 | ACTTGGTTGTCAGGTA[A/G]GTAAGCAAGAAATTCT | rs10748424 |
| 217 | ACTTTACCCTTGTCAA[A/G]TATACCTCCATAGAAA | rs9785216 |
| 218 | AGACTGGCAGGCAATA[A/G]GGACCCCAAAAATGGT | rs1453688 |
| 219 | AGAGAGCTGGAAAATA[A/C]CATGGTTCTTTCTCCT | rs11141918 |
| 220 | AGATAGAGCATTATTG[A/G]AATGGGCTTATTCAAA | rs7700661 |
| 221 | AGCAAATGTAAAGTGT[C/T]AAGGAGATAATGTGAA | rs6095314 |
| 222 | AGCAGACCAAGTGGAA[A/C]TGATTAACCAATCTTA | rs7567280 |
| 223 | AGCCTTACCTGAAGAA[A/G]GGTGTGCAGTATGCAT | rs3091251 |
| 224 | AGCCTTCAAGATTCGG[A/G]ACACAATAAGGATGCC | rs4610242 |
| 225 | AGCTCCCTGTCTTCTC[A/C]GAATAAAACCCAGTGG | rs2139405 |
| 226 | AGGAAATGTAGTTTCA[C/G]GAAAGGAATTTGACAC | rs2031654 |
| 227 | AGGATGGCCTCAGACC[A/G]CGGGAAGGAACTAGAC | rs4730838 |
| 228 | AGGCCTCACAATTAAA[A/G]CTGACTACAAAAAGAA | rs168965 |
| 229 | AGGCGCAATGATTTTA[C/T]GGTAGAGGTGCTTTTG | rs194781 |
| 230 | AGGCTGGGGACTAGTG[C/G]ATCATCGCTTAGGATT | rs4341081 |
| 231 | AGGTTAAGCTGAAAGA[C/T]GCTTGAAACTTCAGAT | rs10816631 |
| 232 | AGTTTGATAATGTAAA[C/T]TGGGGAAAGTGAGTAT | rs10121700 |
| 233 | ATAATAACAAATATGA[C/T]GACTCACTGAGCATTT | rs1594243 |
| 234 | ATACAATCCTCTCCCA[C/T]AGTCACTGTTCAGGAG | rs8034723 |
| 235 | ATATCACAGGTGCCAA[A/G]AACTAAGCATGTCCAA | rs4956126 |
| 236 | ATATGGAAGAAACCAG[A/G]AATAGTGAAATTTGGG | rs7107881 |
| 237 | ATCAGGTCATCTGGAC[A/G]AATCTTGAAACACATT | rs10077522 |
| 238 | ATCGCTGACTGAAAGA[C/T]AGGTCATTTTTAAAAT | rs12147614 |
| 239 | ATCTGATGTATCCTAG[A/T]ACTCTGCAAAACACCA | rs2420378 |
| 240 | ATGGCATCTAAGGACA[C/T]AAGTTGCTACGGAGTA | rs4888286 |
| 241 | ATGTGAAACTGAAGCA[G/T]AGGAAAATACTTACAT | rs6095325 |
| 242 | ATTGTTTTGGTAGGCA[A/C]ACTTTCAGAACTCTTC | rs165914 |
| 243 | CAAATACAAGTCTACA[C/G]AAAACTGAATAAAATT | rs7662513 |
| 244 | GTGTCCATTCCTTAAT[C/T]AGCTAGATCCACCATA | rs1502061 |
| 245 | CAAGTTTATATATCAA[A/G]GTAACCATCACATCCA | rs902581 |
| 246 | CACACATGTATAGACA[C/G]ACATACAACCTTCCCA | rs194782 |
| 247 | TCGATGGCATTTTAGC[G/T]CCTCTGTTATTGTCTA | rs10508010 |
| 248 | CACATGTGTCACCTCA[C/T]GTGATTCTCACCACAG | rs1047929 |
| 249 | CACGGCCCCCCGATGC[A/G]AACATTGCCAGGCCCT | rs7660550 |
| 250 | CAGGATATTGGACACA[A/T]CTGAAGAGTAAATTCA | rs7765427 |
| 251 | CAGTTCTGGAATAGAG[A/C]ATATTGATTTATGACA | rs1404868 |
| 252 | CATCTATAAGTGTCGG[C/T]CACAAGCATCTACTAC | rs720727 |
| 253 | CATGTTTGTAAAGAAA[A/G]TAGGGCTAATGAGGAA | rs6827845 |
| 254 | CATTTGTTCTTGACAC[A/G]GCCATGCAAATGTTAG | rs10269174 |
| 255 | CCAGTTTGAATGAAAA[A/C]CATCACTGAAATAATC | rs1032042 |
| 256 | CCATGCCCGACAATCA[G/T]ACACTTTTACATCACC | rs7628779 |
| 257 | CCATGTGGTAGCAATA[G/T]CCTTACAATTTTCGCT | rs1950070 |
| 258 | CCCAACTTGGAGATAA[A/C]CATTGTTAAATATGAA | rs831663 |
| 259 | CCCACTACTGAAGGAA[A/G]GGGGAGTATACCGTAT | rs2319231 |
| 260 | CCCCAAAGACAACCTA[C/G]ATGAGTGAAGAGCCAA | rs7067701 |
| 261 | CCCCAGCATGTCCCGC[A/G]AAGTGCTTTTCATGTG | rs17781350 |
| 262 | TTCTTGGATCATTTGG[A/C]AATGTGAAAGATCTAC | rs862480 |
| 263 | CCTCAGTGGCTGAAGA[G/T]CTATGTCCAGGGAGCA | rs2304496 |
| 264 | CCTGTGTCTAGAAATA[A/G]CTTCATAGAAGAAGAC | rs3826176 |
| 265 | CGTCTAACTAACAACA[C/G]TGTGGTATTAGTGCCT | rs17309796 |
| 266 | CTCTCACCTTATAAAG[A/G]AGCGACAGAAAGAATG | rs6909578 |
| 267 | CTCTCCTCCTCACGCA[A/G]GTTCGACCTTAGCTCA | rs6944993 |
| 268 | CTCTCTTCTCCCTATC[A/G]ACCTTCTTCTCAGCCA | rs7896663 |
| 269 | CTCTTATCAGTGGCTA[C/T]AATGACTATGAGAAAA | rs6905587 |
| 270 | CTGAAGTCCTTGCTGC[C/T]AGTTCAAACTTGTTCA | rs7317235 |
| 271 | CTGAGTATCTAACCAT[A/G]AAACTAGAGGTGGAGT | rs4710333 |
| 272 | CTGTACACCATAAATA[C/T]GTATCTTTCTTGTTTT | rs13382085 |
| 273 | CTTGTTTTTAAATCCT[A/G]AAGTCATGTCAGTTTT | rs17104962 |
| 274 | CTTTTCACTATCATCA[C/T]GAAATTCCTTTGCTTC | rs1507214 |
| 275 | CTTTTCCTTATCCAGC[A/G]ATGACTTACCTCTTAC | rs569694 |
| 276 | GAAAACAGACTCATTC[A/G]CCAACAAATAATGGAT | rs1333996 |
| 277 | GAACAAAAGCACATAG[C/T]ATCTTAAATGCAGTTT | rs11532826 |
| 278 | GAACTTCAAAATTCCA[A/G]GGTGCAAATATATTGT | rs11163592 |
| 279 | GAAGATGATTACCCAA[A/G]CCATCAGGTACACAGA | rs9688196 |
| 280 | GACACTAGACATTGGG[A/T]ATGAACAGACATCAGG | rs2699749 |
| 281 | GACTTGTTTGGTAAAG[A/T]ATTGGCAGAGTATCAT | rs9323049 |
| 282 | GAGAACATTTAGCTCA[A/G]GTCTAATAGCAGTTAT | rs3914604 |
| 283 | GAGCTGTAAATATGTA[C/T]AATGTGAAGCTGATAG | rs6766592 |
| 284 | GAGGCTCTGCATCCAG[G/T]AATCCAGGCTTCTAGT | rs16973718 |
| 285 | GATACACAGATTGTCA[C/T]TAGGTTATCTTCTGTA | rs4797877 |
| 286 | GATGTATATTATCTCA[A/G]AGACTTACTAGTTTAG | rs10016434 |
| 287 | GATTACATTTTGCTTC[A/G]AGTGCCTCAGGGCTCA | rs11853033 |
| 288 | GATTGCAAAGGGTGGA[C/T]GAAGATAGGAGAGAGG | rs982655 |
| 289 | GCAAATGCTTCACAAC[A/T]GTTGTCCTTGGTCTAA | rs6427673 |
| 290 | GCAATCACATGTATCA[G/T]CTGGAGGTCTCACTAG | rs10851142 |
| 291 | GCAGAGGATCCAAAGT[A/G]AACCCAAGCTATTCTC | rs11941453 |
| 292 | GCCAAAATTTGGGCGC[A/C]GATTCCACCACTTGGT | rs3917768 |
| 293 | GCGTGACCAGGTAGAA[C/T]TAGCCTCAGCATGGAA | rs3091252 |
| 294 | GCGTTAGAATTAGAGA[C/G]GAGCCTGTGACGCTTG | rs2171304 |
| 295 | GCTCTAAAGAAACAGC[A/G]CTTGCCTGGGCTTGGC | rs6582553 |
| 296 | GCTGGACATGGATTAC[G/T]ACTTTCGCACTTTAAC | rs1883881 |
| 297 | GCTTCTCTTTTTCACG[C/T]CCTCAAGCCTGAAAAT | rs2372413 |
| 298 | GCTTTAAACCATGACC[A/C]AAATTTAAGGTCTAGA | rs166412 |
| 299 | GGAAAGTTCAGTATCG[C/G]ATTAATAAAGAGAAAC | rs4750774 |
| 300 | GTGGAAATATTTGAAA[A/G]CATTATCCAAGTACAT | rs7577717 |
| 301 | TAATCAAAACTGTATG[A/G]CACTGGCATAAAGAGA | rs2319227 |
| 302 | TAGTTACCCAACATTA[C/T]GCCAAGATCAGTGATT | rs2306041 |
| 303 | TGGAGATCTAAAAATC[A/C]AAATGCATTGAATTTT | rs4526683 |
| 304 | TTATACCATTCTAGAC[A/G]CTAGTCCTTTGTCAGA | rs6449750 |
| 305 | TTCCTGTTTTCACCTC[C/T]ATACGATGATACCAAT | rs12884090 |
| 306 | TTTGAAATACATAACA[C/G]GAACTCAAAAATGAAA | rs9563195 |
| 307 | TAAATTATAAAGCAAA[C/G]TTATAGCCAATTCTCA | rs10482872 |
| 308 | TTTTAAAATGTGCCTA[C/T]GTCCTTCCAACTGTAT | rs1392326 |
| 309 | CAAAATTCTAAAATAT[A/T]AGAGTGCAGTTGTCAT | rs3846820 |
| 310 | AAAATAAAAGGAATCA[A/G]ATTTATCCAGTAGGAT | rs17056214 |
| 311 | AAGGTCAGCACTGTCA[C/T]GTAAGGTATGTGCTGA | rs6588759 |
| 312 | ACAGACAAGCACTAAC[C/T]CAATGTCACAGAGCTC | rs12353211 |
| 313 | AGGGTAACTTGTATAG[A/G]AGAAGTGTCTTTCATT | rs7972470 |
| 314 | GGTAGTAAAATAGGCC[C/T]GAATACAGCAAATCAC | rs9315895 |
| 315 | ATCGTTCCAGTCTTAG[C/T]ATATGTGCTGCCAAAG | rs4869544 |
| 316 | CAAAGATATTCAGGAA[A/C]CAACAAGGTAAAATTT | rs4528272 |
| 317 | CAAATCAGGGCAAAAA[G/T]TCAACAGCTTCTGCCC | rs1389798 |
| 318 | CAACTCCATACCATGC[A/G]AGGGGACCACAGGTGT | rs35979410 |
| 319 | TACCCTCTGACTTTTA[A/G]CCACCTTGATCATTGC | rs1566655 |
| 320 | CCAAACTTGCAACTCA[C/T]ATCTGAAATTGGAAGG | rs9634125 |
| 321 | CCATGCCAACCGGTGC[G/T]CTGCCAACACCACTGC | rs7302914 |
| 322 | CCTGAGGGGACATTAG[A/G]ATTTTCTTCCTGGATT | rs1500422 |
| 323 | GAAAGTGCCAGGGGAA[C/G]TGCAACTTGGCAATGG | rs2851870 |
| 324 | GAGAGTTCTGCCAGAA[A/G]CTAAGTTCATTCAAAG | rs11054588 |
| 325 | GCATTTTCCAAACAGA[C/G]GCTGATTGTTTATACT | rs7140726 |
| 326 | GGCCTTTGAGAATTGC[A/G]GAAGGAGCCCACTGGG | rs407929 |
| 327 | CGGACACCTACCTAAC[A/T]ATTCGCCACCCTTCCA | rs9937623 |
| 328 | GTCAAGTCTGAACCTC[A/G]CATTTTCTCCCATCTG | rs3820821 |
| 329 | TACAGTTAGGTAAAGA[C/T]AGAGTGGTGAGGGTAA | rs7304649 |
| 330 | TCTGTTAACGTACCAT[A/G]ATCTGTGCTTCGCTCG | rs11135724 |
| 331 | ATAAAACAATCTTAGG[A/C]CATTGATAAAGAGAAA | rs1552194 |
| 332 | TTACCCTTTCCCTGAG[A/T]AACATTGTACATATCC | rs17171382 |
| 333 | TCTCCTTGTAATTTTC[C/T]ACTGATACATGAGCTT | rs4761127 |
| 334 | AATTACGGTGAAGACA[A/G]GAAAAATGGGAAAGAG | rs1465157 |
| 335 | AGACTCAAAAGACATG[A/G]ATGATCCAAATATGAC | rs2926110 |
| 336 | AGAGCAGCTGGACAAC[C/T]CAAATCAACGCTGGAC | rs6095323 |
| 337 | ATGCATTGAAAATGAC[A/G]AATGAAATCCCTTTAT | rs7973241 |
| 338 | CAAAATGAAATAGAAA[A/C]AACAGAGTAGCCCAAA | rs7559372 |
| 339 | TATATCTTACAAATTA[C/G]GAAGTTCTTTGTAGGT | rs1502063 |
| 340 | CAAGCTTGTGCTGCTA[C/G]AACTCAAGGGACTCTG | rs2290891 |
| 341 | GAGTCTAATATGGTCA[A/G]GTGTTAAAATTCTTTC | rs7149994 |
| 342 | CCCGTAAGTCACAAAC[C/T]GGCCGCATGGCCCTTG | rs6446605 |
| 343 | CCCTTTTCATGCTACT[C/T]AACATTACGAATCTCA | rs16906923 |
| 344 | CCTTGCTGCTCAGGAA[C/T]AATCATTGGAACTCCA | rs16988954 |
| 345 | CTGAAAGATAACAAAA[G/T]GCGTGCTCATGGGAAA | rs7714857 |
| 346 | GAGCGAGGAAGGCAGA[C/T]GAGAGGATCTTTCCAG | rs7692314 |
